SEQUENCE LISTING (10 sequences)

SEQ ID NO: 1 - protein, 20 aa, Artificial Sequence
  Description of Artificial Sequence Synthetic peptide
GSGSSRGGSGSGGSGGGGSK

SEQ ID NO: 2 - protein, 14 aa, Artificial Sequence
  Description of Artificial Sequence Synthetic peptide
  MOD_RES (6)..(6): A or S
GSTSGXGKSSEGKG

SEQ ID NO: 3 - protein, 18 aa, Artificial Sequence
  Description of Artificial Sequence Synthetic peptide
GSTSGSGKPGSGEGSTKG

SEQ ID NO: 4 - protein, 7 aa, Artificial Sequence
  Description of Artificial Sequence Synthetic peptide
GGGGGGS

SEQ ID NO: 5 - protein, 15 aa, Artificial Sequence
  Description of Artificial Sequence Synthetic peptide
GGGGSGGGGSGGGGS

SEQ ID NO: 6 - DNA, 1611 bp, Artificial Sequence
  Description of Artificial Sequence Synthetic polynucleotide
atgtgccatcagcaacttgtcatctcctggttctccctcgtgttcctggcctcccctcttgtcgcgatttgggagctgaagaaagatgtgtacgtcgtggaactcgactggtacccggacgcccccggggaaatggtggtgctcacttgtgatactcccgaagaggatggaattacctggaccctcgatcagtcctccgaggtcttgggatccggcaaaactctgaccatccaagtcaaggaattcggcgacgcggggcagtacacctgtcacaagggcggagaagtgctgtcgcactcactcctgctccttcacaaaaaggaggacggcatctggtcgaccgacatcctgaaggaccagaaggaacccaagaacaagacctttctgcgctgcgaggccaagaactattcgggaaggttcacctgttggtggctgactaccatctccaccgacctgactttctccgtgaagtcctctcggggttcgagcgacccgcagggtgttacgtgcggtgctgcaaccctgtccgcggagagagtgcggggggacaacaaggaatacgagtactcagtggaatgccaggaagatagcgcctgccctgccgccgaagagtccctgccgattgaagtcatggtggacgcagtgcataagttgaaatatgagaactacacctcgtcgttcttcatccgggacatcatcaagcctgacccccctaagaatctgcagctcaagcccctcaagaactccagacaggtcgaagtgtcctgggagtacccagatacgtggagcacaccgcactcgtacttctccttgaccttctgcgtccaagtgcagggaaagtccaaacgggagaagaaggaccgcgtgttcactgataagacttccgctactgtgatctgccgcaaaaacgccagcatcagcgtgcgcgcgcaagatagatactactcaagctcttggtccgaatgggcgtccgtgccatgctcggtgcccggcgtgggcgtgcctggagtgggagcccggaacttgccggtggccacccctgaccccggaatgttcccttgcctgcaccactcccaaaaccttctgagggctgtgtccaacatgctgcagaaggctcggcagaccctggaattctacccctgcacctccgaggagatcgaccacgaagatattaccaaggacaagacctcaaccgtggaagcctgcctgcccctggaactgaccaagaacgaatcgtgcctgaatagccgggaaacctccttcatcaccaacggctcctgcctggcctcacgaaagaccagctttatgatggccctgtgcctgagctcgatctacgaggacctgaagatgtaccaggtcgagttcaagactatgaacgccaagctgctgatggatccgaagcggcagatcttcttggaccagaatatgctggcagtgatcgacgagctgatgcaggccctcaacttcaactccgagactgtgccgcaaaagtcgagcctggaggaaccggacttctacaagaccaagatcaagttatgtattctcctgcacgcgtttaggattcgcgccgtgaccattgatagagtgatgtcctacctgaacgccagctga

SEQ ID NO: 7 - protein, 536 aa, Artificial Sequence
  Description of Artificial Sequence Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - DNA, 4156 bp, Artificial Sequence
  Description of Artificial Sequence Synthetic polynucleotide
ccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacgccgccaccatgtgccatcagcaacttgtcatctcctggttctccctcgtgttcctggcctcccctcttgtcgcgatttgggagctgaagaaagatgtgtacgtcgtggaactcgactggtacccggacgcccccggggaaatggtggtgctcacttgtgatactcccgaagaggatggaattacctggaccctcgatcagtcctccgaggtcttgggatccggcaaaactctgaccatccaagtcaaggaattcggcgacgcggggcagtacacctgtcacaagggcggagaagtgctgtcgcactcactcctgctccttcacaaaaaggaggacggcatctggtcgaccgacatcctgaaggaccagaaggaacccaagaacaagacctttctgcgctgcgaggccaagaactattcgggaaggttcacctgttggtggctgactaccatctccaccgacctgactttctccgtgaagtcctctcggggttcgagcgacccgcagggtgttacgtgcggtgctgcaaccctgtccgcggagagagtgcggggggacaacaaggaatacgagtactcagtggaatgccaggaagatagcgcctgccctgccgccgaagagtccctgccgattgaagtcatggtggacgcagtgcataagttgaaatatgagaactacacctcgtcgttcttcatccgggacatcatcaagcctgacccccctaagaatctgcagctcaagcccctcaagaactccagacaggtcgaagtgtcctgggagtacccagatacgtggagcacaccgcactcgtacttctccttgaccttctgcgtccaagtgcagggaaagtccaaacgggagaagaaggaccgcgtgttcactgataagacttccgctactgtgatctgccgcaaaaacgccagcatcagcgtgcgcgcgcaagatagatactactcaagctcttggtccgaatgggcgtccgtgccatgctcggtgcccggcgtgggcgtgcctggagtgggagcccggaacttgccggtggccacccctgaccccggaatgttcccttgcctgcaccactcccaaaaccttctgagggctgtgtccaacatgctgcagaaggctcggcagaccctggaattctacccctgcacctccgaggagatcgaccacgaagatattaccaaggacaagacctcaaccgtggaagcctgcctgcccctggaactgaccaagaacgaatcgtgcctgaatagccgggaaacctccttcatcaccaacggctcctgcctggcctcacgaaagaccagctttatgatggccctgtgcctgagctcgatctacgaggacctgaagatgtaccaggtcgagttcaagactatgaacgccaagctgctgatggatccgaagcggcagatcttcttggaccagaatatgctggcagtgatcgacgagctgatgcaggccctcaacttcaactccgagactgtgccgcaaaagtcgagcctggaggaaccggacttctacaagaccaagatcaagttatgtattctcctgcacgcgtttaggattcgcgccgtgaccattgatagagtgatgtcctacctgaacgccagctgagaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcaccgttggtttccgtagtgtagtggttatcacgttcgcctaacacgcgaaaggtccccggttcgaaaccgggcactacaaaccaacaacgttaaaaaacaggtcctccccatactctttcattgtacacaccgcaagctcgacaatcatcggattgaagcattgtcgcacacatcttccacacaggatcagtacctgctttcgcttttaaccaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccagcccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttcccctaccggggccgctagc

SEQ ID NO: 9 - protein, 10 aa, Artificial Sequence
  Description of Artificial Sequence Synthetic peptide
VPGVGVPGVG

SEQ ID NO: 10 - protein, 5 aa, Artificial Sequence
  Description of Artificial Sequence Synthetic peptide
  See specification as filed for detailed description of substitutions and preferred embodiments
GGGGS

(19) United States
(12) Patent Application Publication
(10) Pub. No.: US 2024/0148902 A1
(43) Pub. Date: May 9, 2024

(54) COMBINATION GENE THERAPY FOR TREATMENT OF METASTATIC CANCER

(71) Applicants: ENGENE, INC., Montreal (CA); Marie-Line GOULET, Montreal (CA); Jose LORA, Montreal (CA); Shauna DAUPHINEE, Montreal (CA)

(72) Inventors: Marie-Line GOULET, Lachine (CA); Jose LORA, Boston, MA (US); Shauna DAUPHINEE, Pointe-Claire (CA)

(21) Appl. No.: 18/277,944

(22) PCT Filed: Feb. 18, 2022
(86) PCT No.: PCT/US22/17099

§ 371 Date: Aug. 18, 2023

Related U.S. Application Data
(60) Provisional application No. 63/150,846, filed on Feb. 18, 2021.

(51) Int. Cl. A61K 48/00; A61K 9/00; A61P 35/00; C07K 14/54; C12N 15/117

(52) U.S. Cl. CPC A61K 48/005; A61K 9/0019; A61K 48/0041; A61P 35/00; C07K 14/5434; C12N 15/117; C12N 2310/17

(57) ABSTRACT

The present disclosure relates to methods and compositions for the localized expression of IL-12, preferably in combination with an RIG-I agonist, for activating a memory T cell response to a cancer antigen. In embodiments, the method is effective for treating metastatic disease.

### CROSS-REFERENCE TO RELATED APPLICATIONS

This application claims the benefit of priority to U.S. Provisional Application No. 63/150,846, filed Feb. 18, 2021, the contents of which are hereby incorporated by reference in their entirety and for all purposes.

### FIELD OF THE INVENTION

The present disclosure relates to methods and compositions for treating tumor metastasis at a distant site, by the localized delivery and expression of IL-12, preferably in combination with a type I IFN (IFN-1) activator/inducer.

### BACKGROUND OF THE INVENTION

Cancerous diseases and tumors are among the major causes for human deaths and severe illness. Tumor metastasis in particular, is a major contributor to the deaths of cancer patients mainly due to the ineffectiveness of current therapies once metastases begin to form.

Treating metastatic cancer, especially when it has spread to several different locations in the body, is an enormous challenge. Typically, people with metastatic cancer are treated only with systemic therapies meant to kill cancer cells anywhere in the body. Unfortunately, however, the effectiveness of this approach is far from ideal. Thus, the terms “cure” and “metastatic cancer” are rarely used together. Patients with metastatic tumors are often unresponsive to existing therapies, and achieving long-term remission in these patients is far less likely than it is for patients with localized cancer. Instead, the goal of treatment for metastatic disease is typically to slow the growth of the cancer or to relieve symptoms caused by it.

The reasons metastatic cancer is difficult to treat are not precisely understood, but it is clear that metastatic tumor cells can adapt quickly and become resistant to treatment. In some cases, each metastatic tumor may be growing in a different organ. This makes treatment a challenge because each tumor may have a unique tumor microenvironment and may respond differently to the treatment. Therefore, the prognosis for people with metastatic cancer is generally poor, and metastatic cancer accounts for most cancer deaths.

Accordingly, there remains a need in the art for methods for inhibiting tumor cell growth at a second tumor site distinct from a primary cancer and for treating or suppressing tumor metastasis at a site distinct from the primary cancer in an individual having a carcinoma. Fortunately, the present disclosure provides for these and other needs.

### SUMMARY OF THE INVENTION

The present disclosure resolves the still unmet need in the art for inhibiting tumor metastasis at distant sites, by the localized delivery and expression of IL-12 together with a Type I interferon (IFN-1) activator/inducer, e.g. a RIG-I agonist, a STING agonist, and/or a TLR 7/9 agonist, at a primary tumor site. As demonstrated herein for the first time, the subject therapy stimulates a robust immune response against the primary cancer including cytotoxic CD8+ T cells as well as CD4+ memory T cells, with the latter cell population in particular supporting the systemic effects of the subject therapy on distant metastases. In some embodiments, the primary tumor site is a mucosal tissue. In some embodiments, the primary tumor site is other than a mucosal tissue. In preferred embodiments, the subject methods and compositions comprise the co-expression of IL-12 with at least one RIG-I agonist.

In one aspect, the disclosure provides a method for activating a memory T cell response to a cancer antigen. The method comprises contacting a primary cancer with a therapeutically effective amount of a composition comprising a nucleic acid polyplex comprising a cationic polymer and/or lipid, a therapeutic nucleic acid construct encoding interleukin-12 (IL-12), and a therapeutic nucleic acid construct comprising a nucleic acid encoding at least one RIG-I agonist, wherein the therapeutic nucleic acid constructs encoding IL-12 and RIG-I are the same or different nucleic acid constructs.

In some embodiments, the method is effective for treating or suppressing a primary cancer. In embodiments, the primary cancer is selected from a breast cancer, colon cancer, prostate cancer, pancreatic cancer, melanoma, lung cancer, ovarian cancer, kidney cancer, brain cancer, a sarcoma, bladder cancer, vaginal cancer, cervical cancer, stomach cancer, a cancer of the gastrointestinal tract, kidney cancer, liver cancer, thyroid cancer, esophageal cancer, nasal cancer, laryngeal cancer, oral cancer, pharyngeal cancer, retinoblastoma, endometrial cancer, and testicular cancer. In embodiments, the primary cancer is other than a mucosal cancer.

In some embodiments, the method is effective for treating or suppressing metastatic disease at a site distinct from a primary cancer. In embodiments, the primary cancer is selected from a breast cancer, colon cancer, prostate cancer, pancreatic cancer, melanoma, lung cancer, ovarian cancer, kidney cancer, brain cancer, a sarcoma, bladder cancer, vaginal cancer, cervical cancer, stomach cancer, a cancer of the gastrointestinal tract, kidney cancer, liver cancer, thyroid cancer, esophageal cancer, nasal cancer, laryngeal cancer, oral cancer, pharyngeal cancer, retinoblastoma, endometrial cancer, and testicular cancer. In some embodiments, the site distinct from the primary cancer is at one or more of: liver, lung, bone, brain, lymph node, peritoneum, skin, prostate, breast, colon, rectum, and cervix. In some embodiments, the metastatic disease is at two or more sites distinct from the primary cancer.

In some embodiments, the RIG-I agonist is selected from the group consisting of eRNA11a, VA RNA1, eRNA41H, MK4621, SLR10, SLR14, and SLR20, and more preferably selected from the group consisting of eRNA41H, eRNA11a.

In some embodiments, the cationic polymer is selected from the group consisting of polyethyleneimine (PEI), PAMAM, polylysine (PLL), polyarginine, chitosan, and derivatives thereof. In some embodiments, the cationic polymer comprises a derivatized chitosan, preferably an amino-functionalized chitosan. In some embodiments, the amino-functionalized chitosan comprises arginine and further comprises, or is functionalized with, a hydrophilic polyol. In some embodiments, the hydrophilic polyol is selected from gluconic acid and glucose.

In some embodiments, the nucleic acid polyplex further comprises a reversible coating comprising one or more polyanion-containing block co-polymers having at least one polyanionic anchor region and at least one hydrophilic tail region, preferably wherein the polyanion-containing block co-polymer is a linear diblock and/or triblock co-polymer.

In some embodiments, the therapeutic nucleic acid construct encoding IL-12, comprises SEQ ID NO: 8.

In another aspect, the disclosure provides a method for treating or suppressing tumor metastasis at a site distinct from a primary cancer in an individual having a primary cancer such as, e.g., bladder cancer, wherein the method comprises contacting to the primary cancer with a therapeutically effective amount of a composition comprising a nucleic acid polyplex comprising a cationic polymer and/or lipid, a therapeutic nucleic acid construct encoding interleukin-12 (IL-12), and a therapeutic nucleic acid construct comprising a nucleic acid encoding at least one RIG-I agonist, wherein the therapeutic nucleic acid constructs encoding IL-12 and RIG-I are the same or different nucleic acid constructs.

In embodiments, the primary cancer is a cancer selected from a breast cancer, colon cancer, prostate cancer, pancreatic cancer, melanoma, lung cancer, ovarian cancer, kidney cancer, brain cancer, a sarcoma, bladder cancer, vaginal cancer, cervical cancer, stomach cancer, a cancer of the gastrointestinal tract, kidney cancer, thyroid cancer, esophageal cancer, nasal cancer, laryngeal cancer, oral cancer, pharyngeal cancer, retinoblastoma, endometrial cancer, and testicular cancer. In one embodiment, the primary cancer is a mucosal cancer selected from the group consisting of a gastrointestinal cancer, a nasal or pulmonary cancer, and a genitourinary cancer. In some embodiments, the primary mucosal cancer is a gastrointestinal cancer, selected from the group consisting of an oral cancer, an esophageal cancer, a stomach cancer, a pancreatic cancer, a liver cancer, a colorectal cancer, and a rectal cancer. In some embodiments, the primary mucosal cancer is a nasal or pulmonary cancer selected from the group consisting of a paranasal sinus cancer, an oropharyngeal cancer, a tracheal cancer, and a lung cancer. In some embodiments, the primary mucosal cancer is a genitourinary cancer selected from the group consisting of a bladder cancer, a urothelial cancer, a urethral cancer, a testicular cancer, a kidney cancer, a prostate cancer, a penile cancer, an adrenal cancer, a uterine cancer, a cervical cancer, and an ovarian cancer. In some embodiments, the genitourinary cancer is bladder cancer.

In some embodiments, the tumor metastatic site is at one or more of: liver, lung, bone, brain, lymph node, peritoneum, skin, prostate, breast, colon, rectum, and cervix. In some embodiments, the tumor metastasis is at two or more different sites.

In some embodiments, the RIG-I agonist is selected from the group consisting of eRNA11a, VA RNA1, eRNA41H, MK4621, SLR10, SLR14, and SLR20, and more preferably selected from the group consisting of eRNA41H, eRNA11a.

In other embodiments, the cationic polymer is selected from the group consisting of polyethyleneimine (PEI), PAMAM, polylysine (PLL), polyarginine, chitosan, and derivatives thereof. In another embodiment, the cationic polymer comprises a derivatized chitosan, preferably an amino-functionalized chitosan.

In some embodiments, the cationic polymer is an amino-functionalized chitosan that comprises arginine and further comprises, or is functionalized with, a hydrophilic polyol. In some embodiments, the hydrophilic polyol is selected from gluconic acid and glucose.

In some embodiments, the nucleic acid polyplex further comprises a reversible coating comprising one or more polyanion-containing block co-polymers having at least one polyanionic anchor region and at least one hydrophilic tail region, preferably wherein the polyanion-containing block co-polymer is a linear diblock and/or triblock co-polymer.

In some embodiments, therapeutic nucleic acid construct encoding IL-12 comprises SEQ ID NO: 8.

In some embodiments, the contacting comprises intravesical instillation. In another embodiment, the administration is oral dosage or intrarectal/intracolonic to gastrointestinal tract (GIT). In some embodiments, the administration is intrarectal/intracolonic administration to the gastrointestinal tract (GIT). In still other embodiments, the contacting is by intratumoral injection. In still other embodiments, the contacting is intranasal or intratracheal administration to the lungs.

Other features, objects and advantages will be apparent from the disclosure that follows.

### BRIEF DESCRIPTION OF THE DRAWINGS

The present disclosure is disclosed with reference to the accompanying drawings, wherein:

FIG. 1 (A) Experimental treatment timeline of female C57BL/6J mice with mEG-70 constructs in an orthotopic model of bladder cancer. Mice bladders were instilled with MB49-Luciferase cells (MB49-Luc; 1×10⁵ cells) at Day 1. Implantation was confirmed by in vivo imaging of luciferase signal at Day 9 post instillation. Mice were distributed equally to treatment groups (n=22) based on the level of bioluminescence and received an intravesical instillation (IVI) of mEG-70 (1 mg DNA/mL; equivalent to 80 μg DNA) on Day 10 (Tx1) and Day 17 (Tx2), with control animals receiving an instillation of 1% mannitol (sham). A cohort of tumor-bearing animals was untreated. Survival was monitored for 85 days. (B) mEG-70-treated animals exhibited long-term survival compared to control mice, of which approximately 70% succumbed to disease. The survival curve for mEG-70 is significantly different from the survival of sham-treated (1% mannitol) or untreated mice (*p<0.05 and **p<0.01, respectively). (C) Mice treated with mEG-70 that demonstrated complete disease regression and did not relapse during the 76-day observation period (referred to as ‘mEG-70 cured’), were re-challenged with MB49-Luc cells to assess protection from recurring disease. In contrast to age-matched naïve controls, which showed robust tumor implantation in 15 out of 17 mice, all mEG-70-cured mice were resistant to tumor recurrence up to 3 weeks after re-challenge (n=17).

FIG. 2 (A) Experimental treatment timeline of female C57BL/6J mice with mEG-70 constructs in an orthotopic model of bladder cancer. Mice bladders were instilled with MB49-Luciferase cells (MB49-Luc; 1×105 cells) on Day 1. Implantation was confirmed by in vivo imaging of luciferase signal at Day 9 post instillation and were distributed equally to treatment groups (n=22) based on the level of bioluminescence. Mice received an intravesical instillation (IVI) of mEG-70 (1 mg DNA/mL; equivalent to 80 μg DNA) on Day 10 (Tx1) and Day 17 (Tx2), with control animals receiving an instillation of 1% mannitol (sham). A cohort of tumor-bearing animals was untreated. Survival was monitored until all mice succumbed to bladder cancer or were considered tumor-free (negative bioluminescence signal, no clinical signs). On Day 85, surviving tumor-free mEG-70-treated mice and age-matched controls, were re-challenged by IVI of MB49-Luc cells (1×10⁵ cells). All mEG-70-treated mice remained tumor-free and, on Day 153, were rechallenged subcutaneously on the flank with either MB49-Luc (1×10⁵ cells) or B16-F10 cells (1×10⁵ cells). (B) mEG-70-treated animals were protected from distant tumor re-challenge with MB49-Luc cells. Only 1 out of 9 animal showed tumor growth, which was markedly delayed. In contrast, the naïve control cohort had 8/9 mice with tumor growth. (C) Mice were re-challenged with B16-F10 cells to assess the specificity of the response. All mice from the re-challenged and naïve control group showed robust B16-F10 tumor implantation (n=8/group).

FIG. 3 (A) Experimental treatment timeline of female C57BL/6J mice with mEG-70 constructs in an orthotopic model of bladder cancer. MB49-Luciferase cells (MB49-Luc; 1×10⁵ cells) were instilled into female C57BL/6J bladders (12-16 weeks) and implantation was confirmed by in vivo imaging of luciferase signal at Day 9 post instillation (using the Lumina LT IVIS imaging system). Mice were distributed equally to treatment groups (n=20) based on the level of bioluminescence (luciferase negative mice were excluded from the study) and received an intravesical instillation (IVI) of mEG-70 (1 mg DNA/mL; equivalent to 80 μg DNA) on Day 10 (Tx1) and Day 17 (Tx2), with control animals receiving an instillation of 1% mannitol (sham). Survival was monitored until all mice succumbed to bladder cancer or were considered tumor-free (negative bioluminescence signal, no clinical signs; data not shown). On Day 167, surviving tumor-free mEG-70-treated mice, and age-matched naïve controls, were injected intraperitoneally with one of either an isotype control (non-depleted), an anti-CD4 antibody, or an anti-CD8 antibody for 4 consecutive days to establish depletion, and then twice a week to maintain. Mice were re-challenged subcutaneously on the flank with MB49-Luc cells (1×10⁵ cells) after the third depleting antibody injection (Day 170; n=6). Tumors were monitored by measuring with a caliper; tumor volume was calculated using the formula (length×width²/2). (B) Naïve mice that received isotype control antibody (non-depleted) have a growing subcutaneous tumor, while mEG-70-treated animals were all protected from distant tumor re-challenge with MB49-Luc cells. (C) Mice that received anti-CD4 antibody (CD4+ T cell-depleted) have a growing MB49-Luc subcutaneous tumor, whether they were naïve or previously cured by mEG-70 treatment. (D) Naïve mice that received anti-CD8 antibody (CD8+ T cell-depleted) all have a growing subcutaneous tumor, but only 1 out of 6 mEG-70-treated animals had an actively growing tumor.

FIG. 4 (A) Experimental treatment timeline of female C57BL/6J mice with mEG-70 constructs. MB49-Luciferase cells (MB49-Luc; 2.5×10⁵ cells in 100 μL) were implanted subcutaneously onto the right flank of C57BL/6J mice (12-16 weeks) under anesthesia to induce disease. When tumors reached ˜50-200 mm³, mice were randomized to treatment groups (n=10). Mice received direct intratumoral (IT) administration of mEG-70 (0.5 mg DNA/mL in 50 μL; equivalent to 25 μg DNA) on Day 1, 4, 8, 11, 15 and 18 with control animals administered 1% mannitol (sham). A cohort of tumor-bearing animals was untreated. Tumor size was monitored by measuring with a caliper 3 times per week (tumor volume was calculated using the formula (length×width²/2). To confirm that tumors had not relapsed in tumor free mEG-70-cured individuals (mEG-70-′cured′; n=9), bioluminescence imaging of luciferase signal was conducted on Day 70 using the Lumina LT IVIS imaging system. On Day 73, mEG-70-cured and age-matched controls, received subcutaneous implantation of MB49-Luc cells (2.5×10⁵ cells in 100 μL) on the left flank. Tumors were monitored three times per week by measuring with a caliper; tumor volume was calculated using the formula (length×width²/2). (B) Intratumoral (IT) administration of mEG-70 inhibited tumor growth compared to sham-treated mice. (C) mEG-70-′cured′ mice were protected from tumor cell re-challenge on the contralateral flank.

### DETAILED DESCRIPTION

The present disclosure contemplates localized expression of IL-12 and a RIG-I agonist at a primary tumor site, for treatment of metastatic disease at a distant site. Localized gene therapies e.g., at mucosal tissue, such as e.g., intravesical administration to the bladder, aerosolized administration to the lungs, intratumoral injection, and/or oral dosage form to gastrointestinal tract (GIT), present an attractive approach to promote local expression of immunomodulatory proteins while minimizing unwanted systemic side effects. Moreover, as demonstrated herein for the first time, it has been surprisingly found that delivery of a therapeutic nucleic acid comprising IL-12 and a RIG-I agonist using the non-viral vector platform disclosed herein provokes a powerful, systemic, anti-tumor activity including both cytotoxic CD8+ T cells and CD4+ memory T cells that can be used to treat and prevent tumor metastasis at sites distant from the primary tumor.

Without being bound by theory activation of the IL-12 pathway at primary cancer sites by way of the subject disclosure acts on effector CD4+ and CD8+ cells leading to potent anti-tumor as well as anti-angiogenic functions, including the induction of memory T cells, whereas simultaneous or sequential stimulation of the RIG-I pathway results in induction of type-I interferons and IFN-stimulated genes, leading to improved cross-presentation of tumor antigens to CD8+ cytotoxic T cells. In the preferred embodiments described and exemplified herein, these concerted biological mechanisms are combined to produce a surprising and remarkably potent inflammatory response driving robust and durable anti-tumor immune responses, coupling stimulation of innate immune system by the RIG-I agonists to the IL-12-mediated stimulation of the adaptive immune response.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, where indicated, the term “about” indicates the designated value±one standard deviation of that value.

The term “combinations thereof” includes every possible combination of elements to which the term refers.

The term “memory T cell response” or “induction of memory T cells” as used herein refers to “activation” of the naïve T cell via the coordinated interactions between molecules on the T cell, antigen-presenting cells (APC), and inflammatory cytokine mediators that direct differentiation of the stimulated T cell into an effector appropriate for the immunological insult e.g., cancer antigen, being addressed. Memory T cell response is known in the art see e.g., Pennock et al (2013) Adv Physiol Educ. 37(4): 273-283; Sprent et al. (2011) Nat Immunol. 12:478-84; MacLeod et al. (2010) Immunology 130(1): 10-15.

Accordingly, “activating a memory T cell response” refers to the activation and programming of T cells from their naïve/resting state to produce a T cell that is capable of mediating immune protection.

The term “cancer antigen” or “tumor antigen” as used herein, refers to a protein produced in a tumor cell that can act as a tumor antigen. “Cancer antigens” or “tumor antigens” are known in the art. For example, the Cancer Epitope Database and Analysis Resource (CEDAR), provides a comprehensive collection of cancer epitopes curated from the literature, as well as cancer epitope prediction and analysis tools see e.g., Koşaloğlu-Yalçin1 et al. (2021) Front. Immunol. 12: 1-14. Exemplary cancer antigens are also disclosed e.g., in the Cancer Antigenic Peptide Database available on the world wide web at caped.icp.ucl.ac.be/Peptide/list.

The term “primary tumor” or “primary cancer” as used herein, refers to a tumor present at the anatomical site where tumor progression began and proceeded to yield a cancerous mass. Exemplary primary cancers include, but are not limited to a primary tumors of the bladder, the colon, the lung, the vagina, the ovaries, the cervix, the kidney, the stomach, gastrointestinal tract, the prostate, the brain, the breast, the pancreas, the lung, the thyroid, the endometrium, the esophagous, the larynx, nasal cancer, oral cancer, melanoma, pharyngeal cancer, retinoblastoma, testicular cancer, etc.

Methods disclosed herein are useful for activating a strong memory T cell response to an antigen e.g., a cancer antigen, such that a cancerous lesion or tumor can be suppressed or cured. Furthermore, the methods disclosed herein that activate a strong memory T cell response to a cancer antigen, result in a durable systemic immunity such that the method is effective for treating or suppressing metastatic disease at a site distinct from a primary cancer.

The term “metastatic” as used herein refers to a tumor that develops at a site away from the site of a primary tumor.

The term “metastatic disease” as used herein, refers to a state or condition which can spread a tumor to another organ or tissue (or part thereof) to another non-adjacent organ or tissue (or part thereof). In an embodiment, the metastatic disease refers to a cancer metastatic disease, e.g. the establishment of metastases. Some cancer cells can acquire the ability to penetrate the walls of lymphatic and/or blood vessels, after which they are able to circulate through the bloodstream (circulating tumor cells) to other sites and tissues in the body. This process is usually known (respectively) as lymphatic or hematogenous spread. After the tumor cells come to rest at another site, they re-penetrate through the vessel or walls, continue to multiply, and eventually another clinically detectable tumor is formed. This new tumor is known as a metastatic (or secondary or tertiary) tumor. When tumor cells metastasize, the new tumor is called a secondary or metastatic tumor a “metastases” or “metastatic disease,” and its cells are like those in the original, primary tumor. This means, for example, that, if bladder cancer metastasizes to the uterus, the secondary tumor is made up of abnormal bladder cells, not of abnormal uterine cells. The tumor in the uterus is then called metastatic bladder cancer, not uterine cancer.

“Metastatic disease” includes, but is not limited to, cancer metastatic spread derived from a cancerous tumor e.g., a mucosal cancer. “Metastatic disease” also includes metastatic spread from benign tumors. Thus, in exemplary embodiments, the metastatic disease includes metastatic spread from cancerous and benign tumors of the breast, colon, prostate, pancreas, skin, lung, ovaries, kidney, brain, bladder, vagina, cervix, stomach, gastrointestinal tract, liver, thyroid, esophagous, nasal cancer, larynx, oral cancer, pharyngeal cancer, retinoblastoma, endometrium, and testicals, etc. In some embodiments, the metastatic disease is a metastatic bladder cancer.

Methods disclosed herein are useful for the prevention or treatment of a metastatic disease by treating or suppressing tumor metastasis at a site distinct from a primary cancer in an individual having a carcinoma. Therefore, as used herein, the expression “prevention or treatment of a metastatic disease” refers to the ability of a composition comprising a nucleic acid polyplex comprising a cationic polymer and/or lipid, and a therapeutic nucleic acid construct encoding interleukin-12 (IL-12), and a therapeutic nucleic acid construct comprising a nucleic acid encoding at least one RIG-I agonist to limit or lower the occurrence of the metastatic disease, limit the metastatic potential of the cancer and/or limit the number and dissemination of the metastases when compared to a control, or to cure the disease. In some embodiments, the methods described herein are useful in the prevention of symptoms associated with a metastatic disease or in limiting the severity of the symptoms associated with a metastatic disease.

The methods described herein can also be useful for limiting the progression of the metastatic disease. As used herein, the expression “limiting the progression of the metastatic disease” refers to the ability of a composition comprising a nucleic acid polyplex comprising a cationic polymer and/or lipid, and a therapeutic nucleic acid construct encoding interleukin-12 (IL-12), and a therapeutic nucleic acid construct comprising a nucleic acid encoding at least one RIG-I agonist, to delay or inhibit the appearance of metastases, limit the number of metastases, limit the size of the metastases and/or limit the number of organs or tissues containing metastases. In an embodiment, the methods described herein can also be useful in preventing the symptoms associated with the progression of metastatic disease or in limiting the severity of the symptoms associated with the progression of metastatic disease.

Thus, “treating” or “treatment” of any disease or disorder refers, in certain embodiments, to ameliorating a disease or disorder that exists in a subject. “Treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder. For example, in an exemplary embodiment, the phrase “treating cancer” refers to inhibition of cancer cell proliferation, inhibition of cancer spread (metastasis), inhibition of tumor growth, reduction of cancer cell number or tumor growth, decrease in the malignant grade of a cancer (e.g., increased differentiation), or improved cancer-related symptoms. Further, as used herein, “treatment” includes preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer.

As used herein, the term “therapeutically effective amount” or “effective amount” refers to an amount of the subject compositions that when administered to a subject is effective to treat a disease or disorder. For example, in an exemplary embodiment, the phrase “effective amount” is used interchangeably with “therapeutically effective amount” or “therapeutically effective dose” and the like, and means an amount of a therapeutic agent that is effective for treating cancer. Effective amounts of the compositions provided herein may vary according to factors such as the disease state, age, sex, weight of the animal.

As used herein, the term “subject” or “individual” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has cancer, an autoimmune disease or condition, and/or an infection that can be treated with an antibody provided herein. In some embodiments, the subject is a human that is suspected to have cancer, an autoimmune disease or condition, and/or an infection.

“Chitosan” is a partially or entirely deacetylated form of chitin, a polymer of N-acetylglucosamine. Chitosans with any degree of deacetylation greater than 50% are used in the present disclosure.

Chitosan may be derivatized by functionalizing free amino groups at the sites of deacetylation. The derivatized chitosans described herein have a number of properties which are advantageous for a nucleic acid delivery vehicle including: they effectively bind and complex the negatively charged nucleic acids, they can be formed into nanoparticles of a controllable size, they can be taken up by the cells and they can release the nucleic acids at the appropriate time within the cells. Chitosans with any degree of functionalization between 1% and 50%. (Percent functionalization is determined relative to the number of free amino moieties on the chitosan polymer prior-to or in the absence of functionalization.) The degrees of deacetylation and functionalization impart a specific charge density to the functionalized chitosan derivative.

A polyol according to the present disclosure may have a 3, 4, 5, 6, or 7 carbon backbone and may have at least 2 hydroxyl groups. Such polyols, or combinations thereof, may be useful for conjugation to a chitosan backbone, such as a chitosan that has been functionalized with a cationic moiety (e.g., a molecule comprising an amino group such as, lysine, ornithine, a molecule comprising a guanidinium group, arginine, or a combination thereof).

The term “C₂-C₆ alkylene” as used herein refers to a linear or branched divalent hydrocarbon radical optionally containing one or more carbon-carbon multiple bonds. For the avoidance of doubt, the term “C₂-C₆ alkylene” as used herein encompasses divalent radicals of alkanes, alkenes and alkynes.

As used herein, unless otherwise indicated, the term “peptide” and “polypeptide” are used interchangeably.

The term “polypeptide” is used in its broadest sense to refer to conventional polypeptides (i.e., short polypeptides containing L or D-amino acids), as well as peptide equivalents, peptide analogs and peptidomimetics that retain the desired functional activity. Peptide equivalents can differ from conventional peptides by the replacement of one or more amino acids with related organic acids, amino acids or the like, or the substitution or modification of side chains or functional groups.

Peptidomimetics may have one or more peptide linkages replaced by an alternative linkage, as is known in the art. Portions or all of the peptide backbone can also be replaced by conformationally constrained cyclic alkyl or aryl substituents to restrict mobility of the functional amino acid sidechains, as is known in the art.

The polypeptides of this disclosure may be produced by recognized methods, such as recombinant and synthetic methods that are well known in the art. Techniques for the synthesis of peptides are well known and include those described in Merrifield, J. Amer. Chem. Soc. 85:2149-2456 (1963), Atherton, et al., Solid Phase Peptide Synthesis: A Practical Approach, IRL Press (1989), and Merrifield, Science 232:341-347 (1986).

As used herein, “linear polypeptide” refers to a polypeptide that lacks branching groups covalently attached to its constituent amino acid side chains. As used herein, “branched polypeptide” refers to a polypeptide that comprises branching groups covalently attached to its constituent amino acid side chains.

The “final functionalization degree” of cation or polyol as used herein refers to the percentage of cation (e.g., amino) groups on the chitosan backbone functionalized with cation (e.g., amino) or polyol, respectively. Accordingly, “α:β ratio,” “final functionalization degree ratio” (e.g., Arginine final functionalization degree: polyol final functionalization degree ratio) and the like may be used interchangeably with the term “molar ratio” or “number ratio.”

Dispersed systems consist of particulate matter, known as the dispersed phase, distributed throughout a continuous medium. A “dispersion” of chitosan nucleic acid polyplexes is a composition comprising hydrated chitosan nucleic acid polyplexes, wherein polyplexes are distributed throughout the medium.

As used herein, a “pre-concentrated” dispersion is one that has not undergone the concentrating process to form a concentrated dispersion.

As used herein, “substantially free” of polyplex precipitate means that the composition is essentially free from particles that can be observed on visual inspection.

As used herein, physiological pH refers to a pH between 6 to 8.

By “chitosan nucleic acid polyplex” or its grammatical equivalents is meant a complex comprising a plurality of chitosan molecules and a plurality of nucleic acid molecules. In a preferred embodiment, the (e.g., dually-) derivatized-chitosan is complexed with said nucleic acid.

The term “polyethylene glycol” (“PEG”) as used herein is intended to mean a polymer of ethylene oxide having repeat units of —(CH₂CH₂—O)— and the general formula of HO—(CH₂CH₂—O)n-H.

The term “monomethoxy polyethylene glycol” (“mPEG”) as used herein is intended to mean a polymer of ethylene oxide having repeat units of —(CH₂CH₂—O)— and the general formula of CH₃—(CH₂CH₂—O)n-H, for example, a PEG capped at one end with a methoxy group.

### I. Metastatic Disease

Metastasis of cancer refers to a spread of cancer cells from one part of the body to nearby tissues, organs or even distant parts of the body. Typically, when cancer spreads from a primary organ to distant organs it is viewed as a systemic disease, and is difficult to control. There are limited treatment options for subjects who develop metastatic disease, and prognosis is typically poor.

Local therapy used to be deemed futile in the presence of metastatic disease. However, it is now understood that in some malignancies (e.g., renal, breast, and prostate) treatment of the primary tumor may reduce mortality despite established metastatic spread (see e.g., Morgan S C, et al. Nat. Rev. Clin. Oncol. 2011 Jun. 7; 8(8):504-6; Sami-Ramzi Leyh-Bannurah et al. (2017) European Urology 72: 118-124). Even so, while treatment directed against the primary tumor might retard progression of existing metastases, it typically does not provide a cure.

Fortunately as will be described in detail below, it has been surprisingly found that compositions disclosed herein, delivered locally at the site of a primary tumor, provide durable, systemic, and specific anti-tumor immunity.

### II. Compositions

Provided herein are chitosan compositions comprising a chitosan-derivative nucleic acid nanoparticle (polyplex) in complex with a polyanion-containing block co-polymer, e.g. a diblock and/or triblock co-polymer coating, wherein individual polymer molecules comprise a negatively charged anchor region and one or more non-charged hydrophilic tail regions. Exemplary polymer molecules useful in the methods and compositions of the present disclosure are “PEG-PA” polymer molecules comprising a polyethylene glycol (PEG) portion and a polyanion (PA) portion.

### A. Chitosan

The chitosan component of the chitosan-derivative nucleic acid nanoparticle can be functionalized with a cationic functional group and/or a hydrophilic moiety. Chitosan functionalized with two different functional groups is referred to as dually derivatized chitosan (DD-chitosan). Exemplary DD-chitosans are functionalized with both a hydrophilic moiety (e.g., a polyol) and a cationic functional group (e.g., an amino group). Exemplary chitosan derivatives are also described in, e.g., U.S. 2007/0281904; and U.S. 2016/0235863, which are each incorporated herein by reference.

In one embodiment, the dually derivatized chitosan described herein comprises chitosan having a degree of deacetylation of at least 50%. In one embodiment, the degree of deacetylation is at least 60%, more preferably at least 70%, more preferably at least 80%, more preferably at least 90%, and most preferably at least 95%. In a preferred embodiment, the dually derivatized chitosan described herein comprises chitosan having a degree of deacetylation of at least 98%.

The chitosan derivatives described herein have a range of average molecular weights that are soluble at neutral and physiological pH, and include for the purposes of this disclosure molecular weights ranging from 3-110 kDa. Embodiments described herein feature lower average molecular weight of derivatized chitosans (<25 kDa, e.g., from about 5 kDa to about 25 kDa), which can have desirable delivery and transfection properties, and are small in size and have favorable solubility. A lower average molecular weight derivatized chitosan is generally more soluble than one with a higher molecular weight, the former thus producing a nucleic acid/chitosan complex that will release more easily the nucleic acid and provide increased transfection of cells. Much literature has been devoted to the optimization of all of these parameters for chitosan-based delivery systems.

An ordinarily skilled artisan will recognize that chitosan refers to a plurality of molecules having a structure of Formula I, wherein n is any integer, and each R1 is independently selected from acetyl or hydrogen, wherein the degree of R1 selected from hydrogen is between 50% to 100%. Also, chitosan referred to as having an average molecular weight, e.g., of 3 kD to 110 kD, generally refers to a plurality of chitosan molecules having a weight average molecular weight of, e.g., 3 kD to 110 kD, respectively, wherein each of the chitosan molecules may have different chain lengths (n+2). It is also well recognized that chitosan referred to as “n-mer chitosan,” does not necessarily comprise chitosan molecules of Formula I, wherein each chitosan molecule has a chain length of n+2. Rather, “n-mer chitosan” as used herein refers a plurality of chitosan molecules, each of which may have different chain lengths, wherein the plurality has an average molecule weight substantially similar to or equal to a chitosan molecule having a chain length of n. For example, 24-mer chitosan may comprise a plurality of chitosan molecules, each having different chain lengths ranging from, e.g. 7-50, but which has a weight average molecular weight substantially similar or equivalent to a chitosan molecule having a chain length of 24.

A dually derivatized chitosan of the disclosure may also be functionalized with a polyol, or a hydrophilic functional group such as a polyol. Without wishing to be bound by theory, it is hypothesized that functionalization with a hydrophilic group such as a polyol which may help to increase the hydrophilicity of chitosan (including Arginine-chitosan) and/or may donate a hydroxyl group. In some embodiments, the hydrophilic functional group of the chitosan-derivative nanoparticles is or comprises gluconic acid. See, e.g., WO 2013/138930. In some embodiments, the hydrophilic functional group of the chitosan-derivative nanoparticles is or comprises glucose. Additionally or alternatively, the hydrophilic functional group can comprise a polyol. See, e.g., U.S. 2016/0235863. Exemplary polyols for functionalization of chitosan are further described below.

The functionalized chitosan derivatives described herein include dually derivatized-chitosan compounds, e.g., cation-chitosan-polyol compounds. In general, the cation-chitosan-polyol compounds are functionalized with an amino-containing moiety, such as an arginine, lysine, ornithine, or molecule comprising a guanidinium, or a combination thereof In certain embodiments, the cation-chitosan-polyol compounds have the following structure of Formula I:

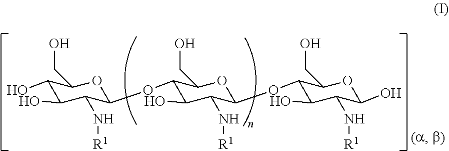

wherein n is an integer of 1 to 650,
α is the final functionalization degree of the cation moiety (e.g., a molecule comprising an amino group such as, lysine, ornithine, a molecule comprising a guanidinium group, arginine, or a combination thereof),
β is the final functionalization degree of polyol; and
each R¹ is independently selected from hydrogen, acetyl, a cation (e.g., arginine), and a polyol.

Preferably, a dually derivatized chitosan of the disclosure may be functionalized with the cationic amino acid, arginine.

In one embodiment, the chitosan-derivative nanoparticle comprises chitosan coupled with gluconic acid at a final functionalization degree of 1%, 2%, 4%, 7%, 8%, 10%, 15%, 20%, 25%, 30%, or greater. In one embodiment, the chitosan-derivative nanoparticle comprises chitosan coupled with glucose at a final functionalization degree of 1%, 2%, 4%, 7%, 8%, 10%, 15%, 20%, 25%, 30%, or greater. In one embodiment, the chitosan derivative nanoparticle comprises chitosan coupled with a cationic moiety (e.g., arginine) at a final functionalization degree of from about 1% to about 25%. In one embodiment, the chitosan derivative nanoparticle comprises chitosan coupled with a cationic moiety (e.g., arginine) at a final functionalization degree of from about 10% to about 40%.

In one embodiment, the chitosan derivative nanoparticle comprises chitosan coupled with a cationic moiety (e.g., arginine) at a final functionalization degree of from about 10% to about 35%. In one embodiment, the chitosan derivative nanoparticle comprises chitosan coupled with a cationic moiety (e.g., arginine) at a final functionalization degree of from about 20% to about 35%. In one embodiment, the chitosan derivative nanoparticle comprises chitosan coupled with a cationic moiety (e.g., arginine) at a final functionalization degree of from about 25% to about 35%. In one embodiment, the chitosan derivative nanoparticle comprises chitosan coupled with a cationic moiety (e.g., arginine) at a final functionalization degree of from about 25% to about 30%.

In one embodiment, the chitosan derivative nanoparticle comprises chitosan coupled with a cationic moiety (e.g., arginine) at a final functionalization degree of from about 15% to about 40%. In one embodiment, the chitosan derivative nanoparticle comprises chitosan coupled with a cationic moiety (e.g., arginine) at a final functionalization degree of from about 15% to about 35%. In one embodiment, the chitosan derivative nanoparticle comprises chitosan coupled with a cationic moiety (e.g., arginine) at a final functionalization degree of from about 15% to about 30%. In one embodiment, the chitosan derivative nanoparticle comprises chitosan coupled with a cationic moiety (e.g., arginine) at a final functionalization degree of from about 15% to about 28%.

In one embodiment, the chitosan derivative nanoparticle comprises chitosan coupled with a cationic moiety (e.g., arginine) at a final functionalization degree of from about 10% to about 35%. In one embodiment, the chitosan derivative nanoparticle comprises chitosan coupled with a cationic moiety (e.g., arginine) at a final functionalization degree of from about 10% to about 30%. In one embodiment, the chitosan derivative nanoparticle comprises chitosan coupled with a cationic moiety (e.g., arginine) at a final functionalization degree of from about 10% to about 28%. In one embodiment, the chitosan derivative nanoparticle comprises chitosan coupled with a cationic moiety (e.g., arginine) at a final functionalization degree of about 28%.

In one embodiment, the chitosan-derivative nanoparticle comprises chitosan coupled with gluconic acid at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with gluconic acid at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with gluconic acid at a final functionalization degree of about 10%.

In one embodiment, the chitosan-derivative nanoparticle comprises chitosan coupled with hydrophilic polyol at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with hydrophilic polyol at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with hydrophilic polyol at a final functionalization degree of about 10%.

In one embodiment, the chitosan-derivative nanoparticle comprises chitosan coupled with glucose at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with glucose at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with glucose at a final functionalization degree of about 10%.

In one embodiment, the chitosan-derivative nanoparticle comprises chitosan coupled with cation (e.g., arginine) at a final functionalization degree of from about 2% to about 40% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 5% to about 40% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 7.5% to about 40% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 10% to about 40% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of from about 2% to about 35% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 5% to about 35% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 7.5% to about 35% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 10% to about 35% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of from about 10% to about 30% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 12% to about 30% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 14% to about 30% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 15% to about 30% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of about 25% and hydrophilic polyol (e.g., glucose or gluconic acid) at a final functional degree of from about 7.5% to about 15%. In one embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of from about 7.5% to about 15%. In one embodiment, the chitosan-derivative nanoparticle comprises chitosan coupled with cation (e.g., arginine) at a final functionalization degree of about 25% and hydrophilic polyol (e.g., glucose or gluconic acid) at a final functional degree of from about 5% to about 20%. In one embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of from about 5% to about 20%.

In a preferred embodiment, the chitosan-derivative nanoparticle comprises chitosan coupled with cation (e.g., arginine) at a final functionalization degree of about 14% and hydrophilic polyol (e.g., glucose or gluconic acid) at a final functional degree of about 10%. In a preferred embodiment, the chitosan-derivative nanoparticle comprises chitosan coupled with cation (e.g., arginine) at a final functionalization degree of about 15% and hydrophilic polyol (e.g., glucose or gluconic acid) at a final functional degree of about 12%. In another preferred embodiment, the chitosan-derivative nanoparticle comprises chitosan coupled with arginine at a final functionalization degree of about 14% and glucose at a final functional degree of about 10%. In another preferred embodiment, the chitosan-derivative nanoparticle comprises chitosan coupled with arginine at a final functionalization degree of about 15% and glucose at a final functional degree of about 12%.

In a preferred embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of about 10%. In another preferred embodiment, the chitosan-derivative nanoparticle comprises chitosan coupled with arginine at a final functionalization degree of about 28% and glucose at a final functional degree of about 10%.

In some embodiments, where appropriate, DD-chitosan includes DD-chitosan derivatives, e.g., DD chitosan that incorporate an additional functionalization, e.g., DD-chitosan with an attached ligand. “Derivatives” will be understood to include the broad category of chitosan-based polymers comprising covalently modified N-acetyl-D-glucosamine and/or D-glucosamine units, as well as chitosan-based polymers incorporating other units, or attached to other moieties. Derivatives are frequently based on a modification of the hydroxyl group or the amine group of glucosamine, such as done with arginine-functionalized chitosan. Examples of chitosan derivatives include, but are not limited to, trimethylated chitosan, thiolated chitosan, galactosylated chitosan, alkylated chitosan, PEI-incorporated chitosan, uronic acid modified chitosan, glycol chitosan, and the like. For further teaching on chitosan derivatives, see, e.g., pp.63-74 of “Non-viral Gene Therapy,” K. Taira, K. Kataoka, T. Niidome (editors), Springer-Verlag Tokyo, 2005, ISBN 4-431-25122-7; Zhu et al., Chinese Science Bulletin, December 2007, vol. 52 (23), pp. 3207-3215; and Varma et al., Carbohydrate Polymers 55 (2004) 77-93.

### A. 1. Chitosan Nucleic Acid Polyplex

The chitosan-derivative nanoparticle compositions generally contain at least one nucleic acid molecule, and preferably a plurality of such nucleic acid molecules. Typical nucleic acid molecules comprise phosphorous as a component of the nucleic acid backbone, e.g., in the form of a plurality of phosphodiesters or derivatives thereof (e.g., phosphorothioate). The proportion of cation-functionalized chitosan-derivative to nucleic acid can be characterized by a cation (+) to phosphorous (P) molar ratio, wherein the (+) refers to the cation of the cation-functionalized chitosan-derivative and the (P) refers to the phosphorous of the nucleic acid backbone. Typically, the (+):(P) molar ratio is selected such that the chitosan-derivative-nucleic acid complex has a positive charge in the absence of the polyanion-containing block co-polymer reversible coating. Thus, the (+):(P) molar ratio is generally greater than 1. In preferred embodiments, the (+):(P) molar ratio is greater than 1.5, at least 2, or greater than 2. In certain preferred embodiments, the (+):(P) molar ratio is greater than 2.

In some cases, the (+):(P) molar ratio is, or is about, 3:1. In some cases, the (+):(P) molar ratio is, or is about, 4:1. In some cases, the (+):(P) molar ratio is, or is about, 5:1. In some cases, the (+):(P) molar ratio is, or is about, 6:1. In some cases, the (+):(P) molar ratio is, or is about, 7:1. In some cases, the (+):(P) molar ratio is, or is about, 8:1. In some cases, the (+):(P) molar ratio is, or is about, 9:1. In some cases, the (+):(P) molar ratio is, or is about, 10:1.

In some cases, the (+):(P) molar ratio is from greater than 1 to no more than about 20:1, from about 2 to no more than about 20:1, or from about 2 to no more than about 10:1. In some cases, the (+):(P) molar ratio is from greater than about 2 to no more than about 20:1, or from greater than about 2 to no more than about 10:1. In some cases, the (+):(P) molar ratio is from about 3 to no more than about 20:1, from about 3 to no more than about 10:1, from about 3 to no more than about 8:1, or from about 3 to no more than about 7:1. In some cases, the (+):(P) molar ratio is from about 3 to no more than 20:1, from about 3 to no more than 10:1, from about 3 to no more than 8:1, or from about 3 to no more than 7:1.

In certain embodiments, the (+):(P) molar ratio is 100:1, preferably less than 100:1. For example, in certain embodiments, (+):(P) molar ratio can be from greater than 1 to less than or equal to 100:1. In some cases, the (+):(P) molar ratio can be from greater than 2 to less than or equal to 100:1. In some cases, the (+):(P) molar ratio can be from greater than or equal to 3 to less than or equal to 100:1. In some cases, the (+):(P) molar ratio can be from greater than or equal to 5 to less than or equal to 100:1. In some cases, the (+):(P) molar ratio can be from greater than or equal to 7 to less than or equal to 100:1. In some cases, the (+):(P) molar ratio can be from greater than 2 to less than or equal to 50:1. In some cases, the (+):(P) molar ratio can be from greater than or equal to 3 to less than or equal to 50:1. In some cases, the (+):(P) molar ratio can be from greater than or equal to 5 to less than or equal to 50:1. In some cases, the (+):(P) molar ratio can be from greater than or equal to 7 to less than or equal to 50:1. In some cases, the (+):(P) molar ratio can be from greater than 2 to less than or equal to 25:1. In some cases, the (+):(P) molar ratio can be from greater than or equal to 3 to less than or equal to 25:1. In some cases, the (+):(P) molar ratio can be from greater than or equal to 5 to less than or equal to 25:1. In some cases, the (+):(P) molar ratio can be from greater than or equal to 7 to less than or equal to 25:1.

In some embodiments, the cationic functional group of the chitosan-derivative nanoparticles is or comprises an amino group. Examples of such amino-functionalized chitosan-derivative nanoparticles include, but are not limited to, those containing chitosan that is functionalized with: a guanidinium or a molecule comprising a guanidinium group, a lysine, an ornithine, an arginine, or a combination thereof. In preferred embodiments, the cationic functional group is an arginine. The proportion of amino-functionalized chitosan-derivative to nucleic acid can be characterized by an amino (N) to phosphorous (P) molar ratio, wherein the (N) refers to the nitrogen atom of the amino group in the amino-functionalized chitosan-derivative and the (P) refers to the phosphorous of the nucleic acid backbone. Typically, the N:P molar ratio is selected such that the chitosan-derivative-nucleic acid complex, in the absence of PEG-PA polymer molecules, has a positive charge at a physiologically relevant pH. Thus, the N:P molar ratio is generally greater than 1. In preferred embodiments, the N:P molar ratio is greater than 1.5, at least 2, or greater than 2. In certain preferred embodiments, the N:P molar ration is greater than 2.

In some cases, the N:P molar ratio is, or is about, 3:1. In some cases, the N:P molar ratio is, or is about, 4:1. In some cases, the N:P molar ratio is, or is about, 5:1. In some cases, the N:P molar ratio is, or is about, 6:1. In some cases, the N:P molar ratio is, or is about, 7:1. In some cases, the N:P molar ratio is, or is about, 8:1. In some cases, the N:P molar ratio is, or is about, 9:1. In some cases, the N:P molar ratio is, or is about, 10:1.

In some cases, the N:P molar ratio is from greater than 1 to no more than about 20:1, from about 2 to no more than about 20:1, or from about 2 to no more than about 10:1. In some cases, the N:P molar ratio is from greater than about 2 to no more than about 20:1, or from greater than about 2 to no more than about 10:1. In some cases, the N:P molar ratio is from about 3 to no more than about 20:1, from about 3 to no more than about 10:1, from about 3 to no more than about 8:1, or from about 3 to no more than about 7:1. In some cases, the N:P molar ratio is from about 3 to no more than 20:1, from about 3 to no more than 10:1, from about 3 to no more than 8:1, or from about 3 to no more than 7:1.

In certain embodiments, the N:P molar ratio is 100:1, preferably less than 100:1. For example, in certain embodiments, N:P molar ratio can be from greater than 1 to less than or equal to 100:1. In some cases, the N:P molar ratio can be from greater than 2 to less than or equal to 100:1. In some cases, the N:P molar ratio can be from greater than or equal to 3 to less than or equal to 100:1. In some cases, the N:P molar ratio can be from greater than or equal to 5 to less than or equal to 100:1. In some cases, the N:P molar ratio can be from greater than or equal to 7 to less than or equal to 100:1. In some cases, the N:P molar ratio can be from greater than 2 to less than or equal to 50:1. In some cases, the N:P molar ratio can be from greater than or equal to 3 to less than or equal to 50:1. In some cases, the N:P molar ratio can be from greater than or equal to 5 to less than or equal to 50:1. In some cases, the N:P molar ratio can be from greater than or equal to 7 to less than or equal to 50:1. In some cases, the N:P molar ratio can be from greater than 2 to less than or equal to 25:1. In some cases, the N:P molar ratio can be from greater than or equal to 3 to less than or equal to 25:1. In some cases, the N:P molar ratio can be from greater than or equal to 5 to less than or equal to 25:1. In some cases, the N:P molar ratio can be from greater than or equal to 7 to less than or equal to 25:1.

In a preferred embodiment, the subject polyplexes have amine to phosphate (N/P) ratio of 2 to 100, e.g., 2 to 50, e.g., 2 to 40, e.g., 2 to 30, e.g., 2 to 20, e.g., 2 to 5. Preferably, the N/P ratio is inversely proportional to the molecular weight of the chitosan, i.e., a smaller molecular weight (e.g., dually) derivatized-chitosan requires a higher N/P ratio, and vice versa.

A nucleic acid of the present disclosure will generally contain phosphodiester bonds, although in some cases nucleic acid analogs are included that may have alternate backbones or other modifications or moieties incorporated for any of a variety of purposes, e.g., stability and protection. Other analog nucleic acids contemplated include those with non-ribose backbones. In addition, mixtures of naturally occurring nucleic acids, analogs, and both can be made. The nucleic acids may be single stranded or double stranded or contain portions of both double stranded or single stranded sequence. Nucleic acids include but are not limited to DNA, RNA and hybrids where the nucleic acid contains any combination of deoxyribo- and ribo-nucleotides, and any combination of bases, including uracil, adenine, thymine, cytosine, guanine, inosine, xanthanine, hypoxanthanine, isocytosine, isoguanine, etc. Nucleic acids include DNA in any form, RNA in any form, including triplex, duplex or single-stranded, anti-sense, siRNA, ribozymes, deoxyribozymes, polynucleotides, oligonucleotides, chimeras, microRNA, and derivatives thereof. Nucleic acids include artificial nucleic acids, including but not limited to, peptide nucleic acid (PNA), phosphorodiamidate morpholino oligo (PMO), locked nucleic acid (LNA), glycol nucleic acid (GNA) and threose nucleic acid (TNA). It will be appreciated that, for artificial nucleic acids that do not comprise phosphorous, an equivalent measure of the (+):P or N:P ratio can be approximated by the number of nucleotide (or nucleotide analog) bases.

In a preferred embodiment, the polyplexes of the compositions comprise chitosan molecules having an average molecular weight of less than 110 kDa, more preferably less than 65 kDa, more preferably less than 50 kDa, more preferably less than 40 kDa, and most preferably less than 30 kDa before functionalization. In some embodiments, polyplexes of the compositions comprise chitosan having an average molecular weight of less than 15 kDa, less than 10 kDa, less than 7 kDa, or less than 5 kDa before functionalization.

In a preferred embodiment, the polyplexes comprise chitosan molecules having on average less than 680 glucosamine monomer units, more preferably less than 400 glucosamine monomer units, more preferably less than 310 glucosamine monomer units, more preferably less than 250 glucosamine monomer units, and most preferably less than 190 glucosamine monomer units. In some embodiments, the polyplexes comprise chitosan molecules having on average less than 95 glucosamine monomer units, less than 65 glucosamine monomer units, less than 45 glucosamine monomer units, or less than 35 glucosamine monomer units.

Chitosan, and (e.g., dually) derivatized-chitosan nucleic acid polyplexes may be prepared by any method known in the art, including but not limited to those described herein.

### A.2 Nucleic Acids

As described above, the chitosan polyplexes can contain a plurality of nucleic acids. In one embodiment, the nucleic acid component comprises a therapeutic nucleic acid. The subject (e.g., dually) derivatized-chitosan nucleic acid polyplexes are amenable to the use of any therapeutic nucleic acid known in the art including, e.g., nucleic acids encoding therapeutic proteins such as hormones, enzymes, cytokines, chemokines, antibodies, mitogenic factors, growth factors, differentiation factors, factors influencing cell apoptosis, factors influencing inflammation, factors influencing the immune response (e.g. immunostimulators), and the like.

A therapeutic nucleic acid may be used to effect genetic therapy by serving as a replacement or enhancement for a defective gene or to compensate for lack of a particular gene product, by encoding a therapeutic product. A therapeutic nucleic acid may also inhibit expression of an endogenous gene. A therapeutic nucleic acid may encode all or a portion of a translation product, and may function by recombining with DNA already present in a cell, thereby replacing a defective portion of a gene. It may also encode a portion of a protein and exert its effect by virtue of co-suppression of a gene product.

In some embodiments, the nucleic acid component comprises a therapeutic nucleic acid construct. The therapeutic nucleic acid construct is a nucleic acid construct capable of exerting a therapeutic effect. Therapeutic nucleic acid constructs may comprise nucleic acids encoding therapeutic proteins, as well as nucleic acids that produce transcripts that are therapeutic RNAs.

In the preferred embodiments described and exemplified herein, the therapeutic nucleic acid construct comprises a nucleic acid encoding IL-12, either alone or in conjunction with an additional immunostimulatory molecule(s). IL-12 is a heterodimeric type 1 cytokine with a four a-helical bundle structure. The active heterodimer, also known as IL-12 p70, comprises 2 subunits encoded by two separate genes, IL-12A (encoding p35) and IL-12B (encoding p40). There are at least 6 splice variant transcripts of IL-12A (ENST00000305579.6, ENST00000466512.1, ENST00000480787.5, ENST00000468862.5, ENST00000496308.1, and ENST00000480088.1). Nucleic and peptide sequences for the human IL-12A isoform 1 precursor are, for example, NM_000882.4, NM_001354582.2, NM_001354583.2, and NP_000873.2, NP_001341511.1, and NP_001341512.1 respectively. Mouse IL12a nucleic and peptide sequences are, for example, NM_001159424.2 and NP_001152896.1, respectively. Human IL-12B genomic sequence, transcript, and peptide sequences are, for example, NG_009618.1, NM_002187.3, and NP_002178.2, respectively. Mouse IL-12B nucleic and peptide sequences are for example, NM_001303244 and NP_001290173.1.

In some embodiments, the single chain IL-12 protein can be generated by fusing the p40 subunit to the p35 subunit through a short amino acid linker sequence. The two subunits can be linked in either the p40-linker-p35 or p35-linker-p40 orientation. The protein can be secreted as a result of the inclusion of the signal peptide from the subunit 5′ of the linker, while the signal peptide is removed from the subunit downstream of the linker sequence. In preferred embodiments, the linker sequence comprises a 10 amino acid sequence derived from bovine elastin and comprised of valine (V), proline (P) and glycine (G) residues (VPGVGVPGVG). In some embodiments, the linker sequence may contain G and/or serine (S) residues, such as (GGGGS)n. In other embodiments, the linker sequence may contain G, S and additional amino acids, including but not limited to, P, arginine (R), lysine (K), threonine (T) and glutamic acid (E). In exemplary embodiments the linker is selected from the group consisting of

(SEQ ID NO: 1)
GSGSSRGGSGSGGSGGGGSK,
(SEQ ID NO: 2)
GSTSG(A/S)GKSSEGKG,
(SEQ ID NO: 3)
GSTSGSGKPGSGEGSTKG,
(SEQ ID NO: 4)
GGGGGGS,
or
(SEQ ID NO: 5)
GGGGSGGGGSGGGGS

In an exemplary embodiment, the nucleic acid sequence encoding hIL-12p40p35 comprises:

(SEQ ID NO: 6)
atgtgccatcagcaacttgtcatctcctggttctccctcgt
gttcctggcctcccctcttgtcgcgatttgggagctgaag
aaagatgtgtacgtcgtggaactcgactggtacccggacg
cccccggggaaatggtggtgctcacttgtgatactcccga
agaggatggaattacctggaccctcgatcagtcctccgag
gtcttgggatccggcaaaactctgaccatccaagtcaagg
aattcggcgacgcggggcagtacacctgtcacaagggcgg
agaagtgctgtcgcactcactcctgctccttcacaaaaag
gaggacggcatctggtcgaccgacatcctgaaggaccaga
aggaacccaagaacaagacctttctgcgctgcgaggccaa
gaactattcgggaaggttcacctgttggtggctgactacc
atctccaccgacctgactttctccgtgaagtcctctcggg
gttcgagcgacccgcagggtgttacgtgcggtgctgcaac
cctgtccgcggagagagtgcggggggacaacaaggaatac
gagtactcagtggaatgccaggaagatagcgcctgccctg
ccgccgaagagtccctgccgattgaagtcatggtggacgc
agtgcataagttgaaatatgagaactacacctcgtcgttc
ttcatccgggacatcatcaagcctgacccccctaagaatc
tgcagctcaagcccctcaagaactccagacaggtcgaagt
gtcctgggagtacccagatacgtggagcacaccgcactcg
tacttctccttgaccttctgcgtccaagtgcagggaaagt
ccaaacgggagaagaaggaccgcgtgttcactgataagac
ttccgctactgtgatctgccgcaaaaacgccagcatcagc
gtgcgcgcgcaagatagatactactcaagctcttggtccg
aatgggcgtccgtgccatgctcggtgcccggcgtgggcgt
gcctggagtgggagcccggaacttgccggtggccacccct
gaccccggaatgttcccttgcctgcaccactcccaaaacc
ttctgagggctgtgtccaacatgctgcagaaggctcggca
gaccctggaattctacccctgcacctccgaggagatcgac
cacgaagatattaccaaggacaagacctcaaccgtggaag
cctgcctgcccctggaactgaccaagaacgaatcgtgcct
gaatagccgggaaacctccttcatcaccaacggctcctgc
ctggcctcacgaaagaccagctttatgatggccctgtgcc
tgagctcgatctacgaggacctgaagatgtaccaggtcga
gttcaagactatgaacgccaagctgctgatggatccgaag
cggcagatcttcttggaccagaatatgctggcagtgatcg
acgagctgatgcaggccctcaacttcaactccgagactgt
gccgcaaaagtcgagcctggaggaaccggacttctacaag
accaagatcaagttatgtattctcctgcacgcgtttagga
ttcgcgccgtgaccattgatagagtgatgtcctacctgaa
cgccagctga.

In an exemplary embodiment, the hIL-12p40p35 amino acid sequence comprises:

(SEQ ID NO: 7)
M C H Q Q L V I S W F S L V F L A S P L V A I
W E L K K D V Y V V E L D W Y P D A P G E M
V V L T C D T P E E D G I T W T L D Q S S E
V L G S G K T L T I Q V K E F G D A G Q Y T
C H K G G E V L S H S L L L L H K K E D G I
W S T D I L K D Q K E P K N K T F L R C E A
K N Y S G R F T C W W L T T I S T D L T F S
V K S S R G S S D P Q G V T C G A A T L S A
E R V R G D N K E Y E Y S V E C Q E D S A C
P A A E E S L P I E V M V D A V H K L K Y E
N Y T S S F F I R D I I K P D P P K N L Q L
K P L K N S R Q V E V S W E Y P D T W S T P
H S Y F S L T F C V Q V Q G K S K R E K K D
R V F T D K T S A T V I C R K N A S I S V R
A Q D R Y Y S S S W S E W A S V P C S V P G
V G V P G V G A R N L P V A T P D P G M F P
C L H H S Q N L L R A V S N M L Q K A R Q T
L E F Y P C T S E E I D H E D I T K D K T S
T V E A C L P L E L T K N E S C L N S R E T
S F I T N G S C L A S R K T S F M M A L C L
S S I Y E D L K M Y Q V E F K T M N A K L L
M D P K R Q I F L D Q N M L A V I D E L M Q
A L N F N S E T V P Q K S S L E E P D F Y K
T K I K L C I L L H A F R I R A V T I D R V
M S Y L N A S Stop.

In another exemplary embodiment, the therapeutic nucleic acid comprises a 4156 bp plasmid DNA (pDNA) (SEQ ID NO: 8) comprised of a codon optimized human interleukin-12 gene termed opt-hIL-12 that encodes a polypeptide having the sequence of SEQ ID NO: 7, linked to a constitutively active cytomegalovirus (CMV) promoter on a NTC9385R backbone with an antibiotic-free selection marker based on sucrose (RNA-OUT). Table 1 shows the 4156 bp plasmid (SEQ ID NO: 8).

TABLE 1

| | |
|---|---|
| CCGCCTAATG AGCGGGCTTT TTTTTGGCTT GTTGTCCACA ACCGTTAAAC | 50 |
| CTTAAAAGCT TTAAAAGCCT TATATATTCT TTTTTTTCTT ATAAAACTTA | 100 |
| AAACCTTAGA GGCTATTTAA GTTGCTGATT TATATTAATT TTATTGTTCA | 150 |
| AACATGAGAG CTTAGTACGT GAAACATGAG AGCTTAGTAC GTTAGCCATG | 200 |
| AGAGCTTAGT ACGTTAGCCA TGAGGGTTTA GTTCGTTAAA CATGAGAGCT | 250 |
| TAGTACGTTA AACATGAGAG CTTAGTACGT ACTATCAACA GGTTGAACTG | 300 |
| CTGATCCACG TTGTGGTAGA ATTGGTAAAG AGAGTCGTGT AAAATATCGA | 350 |
| GTTCGCACAT CTTGTTGTCT GATTATTGAT TTTTGGCGAA ACCATTTGAT | 400 |
| CATATGACAA GATGTGTATC TACCTTAACT TAATGATTTT GATAAAAATC | 450 |
| ATTAGGTACC CCGGCTCTAG TTATTAATAG TAATCAATTA CGGGGTCATT | 500 |
| AGTTCATAGC CCATATATGG AGTTCCGCGT TACATAACTT ACGGTAAATG | 550 |
| GCCCGCCTGG CTGACCGCCC AACGACCCCC GCCCATTGAC GTCAATAATG | 600 |
| ACGTATGTTC CCATAGTAAC GCCAATAGGG ACTTTCCATT GACGTCAATG | 650 |
| GGTGGAGTAT TTACGGTAAA CTGCCCACTT GGCAGTACAT CAAGTGTATC | 700 |
| ATATGCCAAG TACGCCCCCT ATTGACGTCA ATGACGGTAA ATGGCCCGCC | 750 |
| TGGCATTATG CCCAGTACAT GACCTTATGG GACTTTCCTA CTTGGCAGTA | 800 |
| CATCTACGTA TTAGTCATCG CTATTACCAT GGTGATGCGG TTTTGGCAGT | 850 |
| ACATCAATGG GCGTGGATAG CGGTTTGACT CACGGGGATT TCCAAGTCTC | 900 |
| CACCCCATTG ACGTCAATGG GAGTTTGTTT TGGCACCAAA ATCAACGGGA | 950 |
| CTTTCCAAAA TGTCGTAACA ACTCCGCCCC ATTGACGCAA ATGGGCGGTA | 1000 |
| GGCGTGTACG GTGGGAGGTC TATATAAGCA GAGCTCGTTT AGTGAACCGT | 1050 |
| CAGATCGCCT GGAGACGCCA TCCACGCTGT TTTGACCTCC ATAGAAGACA | 1100 |
| CCGGGACCGA TCCAGCCTCC GCGGCTCGCA TCTCTCCTTC ACGCGCCCGC | 1150 |
| CGCCCTACCT GAGGCCGCCA TCCACGCCGG TTGAGTCGCG TTCTGCCGCC | 1200 |
| TCCCGCCTGT GGTGCCTCCT GAACTGCGTC CGCCGTCTAG GTAAGTTTAA | 1250 |
| AGCTCAGGTC GAGACCGGGC CTTTGTCCGG CGCTCCCTTG GAGCCTACCT | 1300 |
| AGACTCAGCC GGCTCTCCAC GCTTTGCCTG ACCCTGCTTG CTCAACTCTA | 1350 |
| GTTCTCTCGT TAACTTAATG AGACAGATAG AAACTGGTCT TGTAGAAACA | 1400 |
| GAGTAGTCGC CTGCTTTTCT GCCAGGTGCT GACTTCTCTC CCCTGGGCTT | 1450 |
| TTTTCTTTTT CTCAGGTTGA AAAGAAGAAG ACGAAGAAGA CGAAGAAGAC | 1500 |
| AAACCGTCGT CGACGCCGCC ACCATGTGCC ATCAGCAACT TGTCATCTCC | 1550 |
| TGGTTCTCCC TCGTGTTCCT GGCCTCCCCT CTTGTCGCGA TTTGGGAGCT | 1600 |
| GAAGAAAGAT GTGTACGTCG TGGAACTCGA CTGGTACCCG GACGCCCCCG | 1650 |
| GGGAAATGGT GGTGCTCACT TGTGATACTC CCGAAGAGGA TGGAATTACC | 1700 |
| TGGACCCTCG ATCAGTCCTC CGAGGTCTTG GGATCCGGCA AAACTCTGAC | 1750 |
| CATCCAAGTC AAGGAATTCG GCGACGCGGG GCAGTACACC TGTCACAAGG | 1800 |
| GCGGAGAAGT GCTGTCGCAC TCACTCCTGC TCCTTCACAA AAAGGAGGAC | 1850 |
| GGCATCTGGT CGACCGACAT CCTGAAGGAC CAGAAGGAAC CCAAGAACAA | 1900 |
| GACCTTTCTG CGCTGCGAGG CCAAGAACTA TTCGGGAAGG TTCACCTGTT | 1950 |
| GGTGGCTGAC TACCATCTCC ACCGACCTGA CTTTCTCCGT GAAGTCCTCT | 2000 |
| CGGGGTTCGA GCGACCCGCA GGGTGTTACG TGCGGTGCTG CAACCCTGTC | 2050 |
| CGCGGAGAGA GTGCGGGGGG ACAACAAGGA ATACGAGTAC TCAGTGGAAT | 2100 |
| GCCAGGAAGA TAGCGCCTGC CCTGCCGCCG AAGAGTCCCT GCCGATTGAA | 2150 |
| GTCATGGTGG ACGCAGTGCA TAAGTTGAAA TATGAGAACT ACACCTCGTC | 2200 |
| GTTCTTCATC CGGGACATCA TCAAGCCTGA CCCCCCTAAG AATCTGCAGC | 2250 |
| TCAAGCCCCT CAAGAACTCC AGACAGGTCG AAGTGTCCTG GGAGTACCCA | 2300 |
| GATACGTGGA GCACACCGCA CTCGTACTTC TCCTTGACCT TCTGCGTCCA | 2350 |
| AGTGCAGGGA AAGTCCAAAC GGGAGAAGAA GGACCGCGTG TTCACTGATA | 2400 |
| AGACTTCCGC TACTGTGATC TGCCGCAAAA ACGCCAGCAT CAGCGTGCGC | 2450 |
| GCGCAAGATA GATACTACTC AAGCTCTTGG TCCGAATGGG CGTCCGTGCC | 2500 |
| ATGCTCGGTG CCCGGCGTGG GCGTGCCTGG AGTGGGAGCC CGGAACTTGC | 2550 |
| CGGTGGCCAC CCCTGACCCC GGAATGTTCC CTTGCCTGCA CCACTCCCAA | 2600 |
| AACCTTCTGA GGGCTGTGTC CAACATGCTG CAGAAGGCTC GGCAGACCCT | 2650 |
| GGAATTCTAC CCCTGCACCT CCGAGGAGAT CGACCACGAA GATATTACCA | 2700 |
| AGGACAAGAC CTCAACCGTG GAAGCCTGCC TGCCCCTGGA ACTGACCAAG | 2750 |
| AACGAATCGT GCCTGAATAG CCGGGAAACC TCCTTCATCA CCAACGGCTC | 2800 |
| CTGCCTGGCC TCACGAAAGA CCAGCTTTAT GATGGCCCTG TGCCTGAGCT | 2850 |
| CGATCTACGA GGACCTGAAG ATGTACCAGG TCGAGTTCAA GACTATGAAC | 2900 |
| GCCAAGCTGC TGATGGATCC GAAGCGGCAG ATCTTCTTGG ACCAGAATAT | 2950 |
| GCTGGCAGTG ATCGACGAGC TGATGCAGGC CCTCAACTTC AACTCCGAGA | 3000 |
| CTGTGCCGCA AAAGTCGAGC CTGGAGGAAC CGGACTTCTA CAAGACCAAG | 3050 |
| ATCAAGTTAT GTATTCTCCT GCACGCGTTT AGGATTCGCG CCGTGACCAT | 3100 |
| TGATAGAGTG ATGTCCTACC TGAACGCCAG CTGAGAATTC CTGTGCCTTC | 3150 |
| TAGTTGCCAG CCATCTGTTG TTTGCCCCTC CCCCGTGCCT TCCTTGACCC | 3200 |
| TGGAAGGTGC CACTCCCACT GTCCTTTCCT AATAAAATGA GGAAATTGCA | 3250 |
| TCGCATTGTC TGAGTAGGTG TCATTCTATT CTGGGGGGTG GGGTGGGGCA | 3300 |
| GGACAGCAAG GGGGAGGATT GGGAAGACAA TAGCAGGCAT GCTGGGGATG | 3350 |
| CGGTGGGCTC TATGGCCCGG GACGGCCGCT AGCACCGTTG GTTTCCGTAG | 3400 |
| TGTAGTGGTT ATCACGTTCG CCTAACACGC GAAAGGTCCC CGGTTCGAAA | 3450 |
| CCGGGCACTA CAAACCAACA ACGTTAAAAA ACAGGTCCTC CCCATACTCT | 3500 |
| TTCATTGTAC ACACCGCAAG CTCGACAATC ATCGGATTGA AGCATTGTCG | 3550 |
| CACACATCTT CCACACAGGA TCAGTACCTG CTTTCGCTTT TAACCAAGGC | 3600 |
| TTTTCTCCAA GGGATATTTA TAGTCTCAAA ACACACAATT ACTTTACAGT | 3650 |
| TAGGGTGAGT TTCCTTTTGT GCTGTTTTTT AAAATAATAA TTTAGTATTT | 3700 |
| GTATCTCTTA TAGAAATCCA AGCCTATCAT GTAAAATGTA GCTAGTATTA | 3750 |
| AAAAGAACAG ATTATCTGTC TTTTATCGCA CATTAAGCCT CTATAGTTAC | 3800 |
| TAGGAAATAT TATATGCAAA TTAACCGGGG CAGGGGAGTA GCCGAGCTTC | 3850 |
| TCCCACAAGT CTGTGCGAGG GGGCCGGCGC GGGCCTAGAG ATGGCGGCGT | 3900 |
| CGGATCGGCC AGCCCGCCTA ATGAGCGGGC TTTTTTTTCT TAGGGTGCAA | 3950 |
| AAGGAGAGCC TGTAAGCGGG CACTCTTCCG TGGTCTGGTG GATAAATTCG | 4000 |
| CAAGGGTATC ATGGCGGACG ACCGGGGTTC GAGCCCCGTA TCCGGCCGTC | 4050 |
| CGCCGTGATC CATGCGGTTA CCGCCCGCGT GTCGAACCCA GGTGTGCGAC | 4100 |
| GTCAGACAAC GGGGGAGTGC TCCTTTTGGC TTCCTTCCCC TACCGGGGCC | 4150 |
| GCTAGC | 4156 |

The R6K origin of replication restricts plasmid replication to a specific strain of Escherichia coli (E. coli). The opt-hIL12 gene encodes the two sub-units (p40 and p35) of the cytokine protein, IL-12. To ensure 1:1 stoichiometry of the subunits, the EG-70 plasmid was designed to contain a single open reading frame (ORF) to monomerize p40 to p35 by the addition of a short repeating elastin linker sequence. The plasmid is also comprised of genes for eRNA11a (an immunostimulatory double-stranded ribonucleic acid [dsRNA]) and adenovirus VA RNA1. The two RNA products of these genes stimulate the RIG-I pathway, which recruits more immune cells to the local tissue. In a further embodiment, this therapeutic nucleic acid is packaged in a dually-derivatized chitosan polymer functionalized with arginine and glucose and coated with a detachable PEG-b-PLE excipients, to form the pharmaceutical composition EG-70. The composition is formulated as an aqueous nanoparticle dispersion in 1% w/w mannitol solution, filter sterilized, lyophilized to a dry powder, and stored at 4° C. The average particle size of the nanoparticle dispersion is in the 75-175 nanometer range.

Therapeutic nucleic acids also include therapeutic DNA in the form of a circular double-stranded DNA plasmid, minicircle DNA (Science Report 6:2315, 2016) or closed-ended linear duplex DNA (Li et al, PLoS One 8(8): e69879, 2013).

Therapeutic nucleic acids also include therapeutic RNAs, which are RNA molecules capable of exerting a therapeutic effect in a mammalian cell. Therapeutic RNAs include, but are not limited to, messenger RNAs, antisense RNAs, siRNAs, short hairpin RNAs, micro RNAs, and enzymatic RNAs. Therapeutic nucleic acids include, but are not limited to, nucleic acids intended to form triplex molecules, protein binding nucleic acids, ribozymes, deoxyribozymes, and small nucleotide molecules. Many types of therapeutic RNAs are known in the art. For example, see Meng et al., A new developing class of gene delivery: messenger RNA-based therapeutics, Biomater. Sci., 5, 2381-2392, 2017; Grimm et al., Therapeutic application of RNAi is mRNA targeting finally ready for prime time? J. Clin. Invest., 117:3633-3641, 2007; Aagaard et al., RNAi therapeutics: Principles, prospects and challenges, Adv. Drug Deliv. Rev., 59:75-86, 2007; Dorsett et al., siRNAs: Applications in functional genomics and potential as therapeutics, Nat. Rev. Drug Discov., 3:318-329, 2004. These include double-stranded short interfering RNA (siRNA).

### A.3 Expression Control Regions

In a preferred embodiment, a polyplex of the disclosure comprises a therapeutic nucleic acid, which is a therapeutic construct, comprising an expression control region operably linked to a coding region. The therapeutic construct produces therapeutic nucleic acid, which may be therapeutic on its own, or may encode a therapeutic protein.

In some embodiments, the expression control region of a therapeutic construct possesses constitutive activity. In a number of preferred embodiments, the expression control region of a therapeutic construct does not have constitutive activity. This provides for the dynamic expression of a therapeutic nucleic acid. By “dynamic” expression is meant expression that changes over time. Dynamic expression may include several such periods of low or absent expression separated by periods of detectable expression. In a number of preferred embodiments, the therapeutic nucleic acid is operably linked to a regulatable promoter. This provides for the regulatable expression of therapeutic nucleic acids.

Expression control regions comprise regulatory polynucleotides (sometimes referred to herein as elements), such as promoters and enhancers, which influence expression of an operably linked therapeutic nucleic acid.

Expression control elements included herein can be from bacteria, yeast, plant, or animal (mammalian or non-mammalian). Expression control regions include full-length promoter sequences, such as native promoter and enhancer elements, as well as subsequences or polynucleotide variants that retain all or part of full-length or non-variant function (e.g., retain some amount of nutrient regulation or cell/tissue-specific expression). As used herein, the term “functional” and grammatical variants thereof, when used in reference to a nucleic acid sequence, subsequence or fragment, means that the sequence has one or more functions of native nucleic acid sequence (e.g., non-variant or unmodified sequence). As used herein, the term “variant” means a sequence substitution, deletion, or addition, or other modification (e.g., chemical derivatives such as modified forms resistant to nucleases).

As used herein, the term “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Some expression control regions confer regulatable expression to an operably linked therapeutic nucleic acid. A signal (sometimes referred to as a stimulus) can increase or decrease expression of a therapeutic nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

Numerous regulatable promoters are known in the art. Preferred inducible expression control regions include those comprising an inducible promoter that is stimulated with a small molecule chemical compound. In one embodiment, an expression control region is responsive to a chemical that is orally deliverable but not normally found in food. Particular examples can be found, for example, in U.S. Pat. Nos. 5,989,910; 5,935,934; 6,015,709; and 6,004,941.

Promoter/enhancer sequences of particular interest include:

| Promoter/enhancer | |
|---|---|
| sequence | Description |
| CMV | Cytomegalovirus immediate early |
| | enhancer and promoter |
| EF1α | Human elongation factor |
| | (EF)-1α promoter |
| CMV/EF1α | CMV enhancer + core |
| | EF1α promoter |
| 2 × CMV/EF1α | 2 × CMV enhancer + core |
| | EF1α promoter |
| CAG | CMV enhancer + promoter, first exon |
| | and first intron of chicken beta-actin |
| | gene + splice acceptor of the rabbit |
| | beta globin gene |
| CMV/EF1α/HTLV | CMV enhancer + Human elongation factor |
| | (EF)-1α promoter + R segment and part |
| | of U5 sequence (R′-U5) of human T-cell |
| | leukemia virus Type 1 Long Terminal Repeat |

In some embodiments of the disclosure, the therapeutic construct is comprised within a plasmid comprising an origin, a multicloning site and a selectable marker. In some embodiments, plasmids of less than 10 kb are desirable. In some embodiments the plasmids used are suitable for gene therapy in human patients, and/or are engineered for high levels of transient gene expression in mammalian tissues. In preferred embodiments, the plasmid is selected from the group consisting of the Nanoplasmid™ (e.g. NTC9385 plasmid, NTC9385R, NTC9385R-RIG-I, NTC9385R (3CpG), NTC₉₃₈₅R-eRNA41H-CpG, NTC8685 plasmid (Nature Technology), gWIZ plasmid (Genlantis), or pVAX1 plasmid (Thermofisher Scientific). See, e.g., U.S. Pat. Nos. 6,027,722, 6,287,863, 6,410,220, 6,573,091, 9,012,226, 9,017,966, 9,018,012, 9,109,012, 9,487,788, 9,487,789, 9,506,082, 9,550,998, 9,725,725, 9,737,620, 9,950,081, 10,047,365, 10,144,935, and 10,167,478. In some embodiments, the plasmid has been “retrofitted” to remove antibiotic selection agents and/or to increase expression levels.

For further teaching, see WO 2008/020318, which is expressly incorporated herein in its entirety by reference. In one embodiment, the nucleic acid of the (e.g., dually) derivatized-chitosan nucleic acid polyplex is an artificial nucleic acid.

In one embodiment, the nucleic acid of the DD-chitosan nucleic acid polyplex is a therapeutic nucleic acid. In one embodiment, the therapeutic nucleic acid is a therapeutic RNA. Preferred therapeutic RNAs include, but are not limited to, antisense RNA, siRNA, short hairpin RNA, micro RNA, and enzymatic RNA.

In one embodiment, the therapeutic nucleic acid is DNA.

In one embodiment, the therapeutic nucleic acid comprises a nucleic acid sequence encoding a therapeutic protein. In one embodiment, the therapeutic protein is IL-12.

### B. Polyols

Chitosan-derivative nanoparticles can be functionalized with a polyol. Polyols useful in the present disclosure in general are typically hydrophilic. In some cases, the chitosan-derivative nanoparticles are functionalized with a cationic component such as an amino group and with a polyol. Such chitosan-derivative nanoparticles functionalized with a cationic moiety such as an amino group and a polyol are referred to as “dually-derivatized chitosan nanoparticles.”

In some embodiments, the chitosan-derivative nanoparticle comprises a polyol of Formula II:

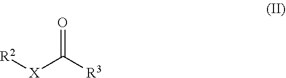

wherein:
R² is selected from: H and hydroxyl;
R³ is selected from: H and hydroxyl; and
X is selected from: C₂-C₆ alkylene optionally substituted with one or more hydroxyl substituents.

In some embodiments, the chitosan-derivative nanoparticle is functionalized with a polyol of Formula II, wherein R² is selected from: H and hydroxyl; R³ is selected from: H and hydroxyl; and X is selected from: C₂-C₆ alkylene optionally substituted with one or more hydroxyl substituents.

In some embodiments, the chitosan-derivative nanoparticle comprises a polyol of Formula III:

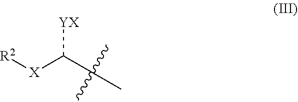

wherein:
[symbol] Y is ═O or —H₂;
R² is selected from: H and hydroxyl;
R³ is selected from: H and hydroxyl;
X is selected from: C₂-C₆ alkylene optionally substituted with one or more hydroxyl substituents; and

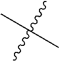

denotes the bond between the polyol and the derivatized chitosan.

In one embodiment, a polyol according to the present disclosure having 3 to 7 carbons may have one or more carbon-carbon multiple bonds. In a preferred embodiment, a polyol according to the present disclosure comprises a carboxyl group. In a further preferred embodiment, a polyol according to the present disclosure comprises an aldehyde group. A skilled artisan will recognize that when a polyol according to the present disclosure comprises an aldehyde group, such polyol encompasses both the open-chain conformation (aldehyde) and the cyclic conformation (hemiacetal).

Non-limiting examples of a polyols include gluconic acid, threonic acid, glucose and threose. Examples of other such polyols, which may have a carboxyl and/or aldehyde group, or may be a saccharide or acid form thereof, are described in more detail in U.S. Pat. No. 10,046,066, the disclosure of which is expressly incorporated by reference herein. A skilled artisan will recognize that the polyols are not limited to a specific stereochemistry.

In a preferred embodiment, the polyol may be selected from the group consisting of 2,3-dihydroxylpropanoic acid; 2,3,4,5,6,7-hexahydroxylheptanal; 2,3,4,5,6-pentahydroxylhexanal; 2,3,4,5-tetrahydroxylhexanal; and 2,3-dihydroxylpropanal.

In a preferred embodiment, the polyol may be selected from the group consisting of D-glyceric acid, L-glyceric acid, L-glycero-D-mannoheptose, D-glycero-L-mannoheptose, D-glucose, L-glucose, D-fucose, L-fucose, D-glyceraldehyde, and L-glyceraldehyde.

In some embodiments, the polyol may be compound of Formula IV or Formula V:

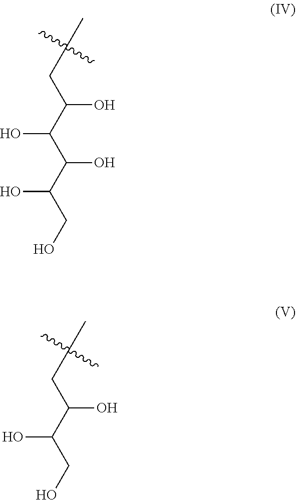 [SMILES: *CC(O)C(O)C(O)C(O)CO ; *CC(O)C(O)CO]

In a preferred embodiment, the polyol is a compound of Formula IV. In some cases, the polyol of Formula IV has been coupled to the chitosan by reductive amination.

A hydrophilic polyol that has a carboxyl group may be coupled to chitosan or a cation functionalized chitosan such as an amine-functionalized chitosan (e.g., Arg-coupled chitosan (Arg-chitosan)). In some embodiments, the polyol is coupled at a reaction pH of 6.0±0.3. At this pH, the carboxylic acid group of the hydrophilic polyol may be attacked by uncoupled amines on the chitosan backbone according to a nucleophilic substitution reaction mechanism.

An ordinarily skilled artisan will recognize that, when coupling such a hydrophilic polyol to Arg-chitosan, it is also possible that a small amount of the hydrophilic polyol may form a covalent bond with an amine group of the Arg through the same mechanism, although it is likely that the nucleophilic substitution reaction will occur predominantly with the amine group of the chitosan backbone.

A hydrophilic polyol that is a natural saccharide may be coupled to chitosan, cation-functionalized chitosan, such as amine-functionalized chitosan (e.g., Arg-coupled chitosan (Arg-chitosan)) using reductive amination followed by reduction with NaCBH₃ or NaBH.

### C. Polymer:Polyplex Compositions

Chitosan polyplexes can be mixed with a plurality of polymers, the polymers comprising a hydrophilic, non-charged portion, and a negatively charged (anionic) portion. As described above, the chitosan polyplexes are formulated to have a positive charge in the absence of, or prior to, complexing with the anionic portion-containing polymer. Thus under suitable conditions, the polymer component will form a reversible charge:charge complex with the chitosan-derivative nucleic acid polyplexes. In some embodiments, the polymers of the polymer component are unbranched. In some embodiments, the polymers are branched. In some cases, the polymer component comprises a mixture of branched and unbranched polymers.

In some embodiments, the polymer component is released from the chitosan polyplex after administration, after entering a cell, and/or after endocytosis. Without wishing to be bound by theory, it is hypothesized that the polyplex:polymer compositions thus formed by complexing polyplex and the anionic portion-containing polymer can provide improved in vitro, in solution, and/or in vivo stability without substantially interfering with transfection efficiency. In some embodiments, the polyplex:polymer compositions thus formed can provide reduced muco-adhesive properties as compared to, e.g., otherwise identical, polyplexes without the polymer component.

In a preferred embodiment, the polyplex:polymer compositions have a low net positive, neutral, or net negative zeta potential (from about +10 mV to about −20 mV) at physiological pH. Such compositions can exhibit reduced aggregation in physiological conditions and reduced non-specific binding to ubiquitous anionic components in vivo. Said properties can enhance migration of such composition (e.g., enhanced diffusion in mucus) to contact the cell and result in enhanced intracellular release of nucleic acid.

In a preferred embodiment, the polyplex:polymer particle compositions have an average hydrodynamic diameter of less than 1000 nm, more preferably less than 500 nm and most preferably less than 200 nm. In certain embodiments, the polyplex:polymer particle compositions have an average hydrodynamic diameter of from 50 nm to no more than 1000 nm, preferably from 50 nm to no more than 500 nm and most preferably from 50 nm to no more than 200 nm. In certain embodiments, the polyplex:polymer particle compositions have an average hydrodynamic diameter of from 50 nm to no more than 175 nm, preferably from 50 nm to no more than 150 nm. In certain embodiments, the polyplex:polymer particle compositions have an average hydrodynamic diameter of from 75 nm to no more than 1000 nm, preferably from 75 nm to no more than 500 nm and most preferably from 75 nm to no more than 200 nm. In certain embodiments, the polyplex:polymer particle compositions have an average hydrodynamic diameter of from 75 nm to no more than 175 nm, preferably from 75 nm to no more than 150 nm. In certain embodiments, the polyplex:polymer particle compositions have an average hydrodynamic diameter of greater than 100 nm and less than 175 nm.

In one embodiment, the polyplex:polymer compositions have a % supercoiled DNA content of 80%, at least 80%, or preferably 90%, more preferably at least 90%.

In one embodiment, the polyplex:polymer compositions have an average zeta potential of between +10 mV to −10 mV at a physiological pH, most preferably between +5 mV to −5 mV at a physiological pH.

The polyplex:polymer compositions are preferably homogeneous in respect of particle size. Accordingly, in a preferred embodiment, the composition has a low average polydispersity index (“PDF”). In an especially preferred embodiment, a dispersion of the polyplex:polymer composition has a PDI of less than 0.5, more preferably less than 0.4, more preferably less than 0.3, yet more preferably less than 0.25, and most preferably less than 0.2.

In some cases, a dispersion of the polyplex:polymer composition exhibits one or more of the foregoing PDI, average zeta potential, % supercoil DNA, or average particle size (nm) or size range after one or more freeze thaw cycles. In some cases, a dispersion of the polyplex:polymer composition exhibits one or more of the foregoing PDI, average zeta potential, % supercoil DNA, or average particle size (nm) or size range after storage in solution for at least 48 h at 4° C. In some cases, a dispersion of the polyplex:polymer composition exhibits one or more of the foregoing PDI, average zeta potential, % supercoil DNA, or average particle size (nm) or size range after storage in solution for at least for 2 weeks, or more at 4° C.

In some cases, a dispersion of the polyplex:polymer composition exhibits one or more of the foregoing PDI, average zeta potential, % supercoil DNA, or average particle size (nm) or size range after lyophilization and rehydration. In some cases, a dispersion of the polyplex:polymer composition exhibits one or more of the foregoing PDI, average zeta potential, % supercoil DNA, or average particle size (nm) or size range after spray drying and rehydration. In some cases, a dispersion of the polyplex:polymer composition exhibits one or more of the foregoing PDI, average zeta potential, % supercoil DNA, or average particle size (nm) or size range when concentrated (e.g., by ultrafiltration such as tangential flow filtration) to a nucleic acid concentration of at least 250 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 1,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 25,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 2,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 5,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 10,000 μg/mL.

In general, the polyplex:polymer compositions described herein, exhibit favorable solution behavior (e.g., stability and/or non-aggregation) as measured by PDI or mean particle size even in the absence of excipients such as lyoprotectants, cryoprotectants, surfactants, rehydration or wetting agents, and the like. In some cases, the polyplex:polymer compositions described herein exhibit favorable solution behavior (e.g., stability and/or non-aggregation) as measured by PDI or mean particle size in physiological fluids or simulated physiological fluids. For example, in some embodiments, the polyplex:polymer compositions described herein are stable in simulated intestinal fluid, in mammalian urine, and/or when stored in a mammalian bladder (e.g., and in contact with urine).

As described above, the polyplex:polymer compositions described herein are preferably substantially size stable in the composition. In a preferred embodiment, a composition of the disclosure comprises polyplex:polymer particles that increase in average diameter by less than 100%, more preferably less than 50%, and most preferably less than 25%, at room temperature for 6 hours, more preferably 12 hours, more preferably 24 hours, and most preferably 48 hours. In a particularly preferred embodiment, a composition of the disclosure comprises polyplex:polymer particles that increase in average diameter by less than 25% at room temperature for at least 24 hours or at least 48 hours.

The polyplex:polymer particles of the subject compositions are preferably substantially size stable under cooled conditions. In a preferred embodiment, a composition of the disclosure comprises polyplex:polymer particles that increase in average diameter by less than 100%, more preferably less than 50%, and most preferably less than 25%, at 2-8 degrees Celsius for 6 hours, more preferably 12 hours, more preferably 24 hours, and most preferably 48 hours.

The polyplex:polymer particles of the subject compositions are preferably substantially size stable under freeze-thaw conditions. In a preferred embodiment, a composition of the disclosure comprises polyplexes that increase in average diameter by less than 100%, more preferably less than 50%, and most preferably less than 25% at room temperature for 6 hours, more preferably 12 hours, more preferably 24 hours, and most preferably 48 hours following thaw from frozen at −20 to −80 degrees Celsius.

In a preferred embodiment, the composition has a nucleic acid concentration greater than 0.5 mg/ml, and is substantially free of precipitated polyplex. More preferably, the composition has a nucleic acid concentration of at least 0.6 mg/ml, more preferably at least 0.75 mg/ml, more preferably at least 1.0 mg/ml, more preferably at least 1.2 mg/ml, and most preferably at least 1.5 mg/ml, and is substantially free of precipitated polyplex. In another preferred embodiment, the composition has a nucleic acid concentration greater than 2 mg/ml, and is substantially free of precipitated polyplex. More preferably, the composition has a nucleic acid concentration of at least 2.5 mg/ml, more preferably at least 5 mg/ml, more preferably at least 10 mg/ml, more preferably at least 15 mg/ml, and most preferably about 25 mg/ml, and is substantially free of precipitated polyplex. In some embodiments, the composition has a nucleic acid concentration from 0.5 mg/mL to about 25 mg/mL, and is substantially free of precipitated polyplex. In some embodiments, the composition has a nucleic acid concentration of ≤about 25 mg/mL, and is substantially free of precipitated polyplex. The compositions can be hydrated. In a preferred embodiment, the composition is substantially free of uncomplexed nucleic acid.

In a preferred embodiment, the polyplex:polymer particle composition is isotonic. Achieving isotonicity, while maintaining polyplex stability, is highly desirable in formulating pharmaceutical compositions, and these preferred compositions are well suited to pharmaceutical formulation and therapeutic applications.

In certain embodiments, the polyplex:polymer particle composition can be uncoated to release all or part of the, e.g., PEG, polymer coat by reducing pH. In certain embodiments, the polymer coat is released by incubating the particle under a pH condition that is below the pKa of the polyanionic anchor region of the polymer. For example, where the polymer coat is polyglutamate, the polymer coat can be released by incubating the particle at a pH below the pKa of polyglutamate, such as a pH of less than about 4.25. In certain embodiments, the polymer coat can be released by incubating the particle under a pH condition that is at least 0.25 pH units or at least 0.5 pH units below the pKa of the polyanion anchor region of the polymer coat.

In certain embodiments, the polyplex:polymer particle composition can be uncoated to release all or part of the, e.g., PEG, polymer coat by subjecting the particle to a high ionic strength.

Without wishing to be bound by theory, it is hypothesized that certain physiological conditions can promote partial (e.g. >5%), substantial (>50%), extensive, (e.g., >90%), or complete (100%) uncoating of reversibly PEGylated chitosan DNA polyplexes described herein. For example, low pH conditions in certain subcellular compartments (e.g., endosome, early endosome, late endosome, or lysosome) can facilitate release of the polymer coat. As another example, certain extracellular conditions can promote partial (e.g., >5%), substantial (>50%), extensive (>90%), or complete (100%) uncoating of reversibly PEGylated chitosan DNA polyplexes described herein. In some cases, the high ionic strength and/or acidic pH conditions typically encountered in certain positions in the alimentary canal can promote partial (e.g. >5%), substantial (>50%), extensive (>90%), or complete (100%) uncoating of reversibly PEGylated chitosan DNA polyplexes described herein.

In certain embodiments, PEGylated polyplexes described herein are formulated for delivery to a cell, tissue, or bodily compartment (e.g., intestine, small intestine, large intestine, colon, lung, or bladder) such that the polyplexes remain PEGylated and thereby facilitate transfection of the target cell. In some embodiments, PEGylated polyplexes described herein partially (e.g. >5%), substantially (>50%), extensively (e.g., >90%), or completely (100%) release the polymer coat after or during entry into the intracellular environment. In certain embodiments, PEGylated polyplexes described herein are formulated for delivery to a cell, tissue or bodily compartment (e.g., intestine, small intestine, large intestine, colon, lung, or bladder) such that the PEGylated polyplexes described herein partially (e.g., >5%), substantially (>50%), extensively (e.g., >90%), or completely (100%) release the polymer coat upon delivery to a cell, tissue or bodily compartment (e.g., intestine, small intestine, large intestine, colon, lung or bladder).

It will be appreciated that anion charge density and/or pKa of the anionic anchor region of a polymer can be adjusted to promote or inhibit release under intended conditions. It will similarly be appreciated that the pH, volume, and ionic strength, and other conditions of the formulation can be adjusted to promote or inhibit release under intended conditions. For example, for delivery to the intestine through the low pH gastric environment, a PEGylated polyplex formulation can be enteric coated and/or delivered in a buffering agent to increase the pH of the gastric environment. Optimized reversibly PEGylated particle compositions can be identified by assaying for stability and transfection efficiency using assays described herein.

The compositions comprising chitosan polyplex complexed with the anionic portion-containing polymer can be characterized by the ratio of cationic functional groups of the (e.g., dually) derivatized-chitosan polyplex (+) to anion moieties of the polymer (−), referred to as the “(+):(−) molar ratio.” This (+):(−) molar ratio can vary from greater than about 1:100 to less than about 10:1.

In certain embodiments, the (+):(−) molar ratio can be from greater than about 1:75 to less than about 8:1. In some cases, the (+):(−) molar ratio can be from greater than 1:10 to less than 10:1. In some cases, the (+):(−) molar ratio can be from, or from about, 1:10 to, or to about, 10:1. In some cases, the (+):(−) molar ratio can be from, or from about, 1:8 to, or to about, 8:1. In certain embodiments, the (+):(−) molar ratio can be from greater than 1:50 to less than about 10:1. In some cases, the (+):(−) molar ratio can be from greater than 1:25 to less than about 10:1. In some cases, the (+):(−) molar ratio can be from greater than 1:10 to less than about 7:1. In some cases, the (+):(−) molar ratio can be from greater than 1:8 to less than about 7:1. In some cases, the (+):(−) molar ratio can be from greater than 1:8 to less than about 6:1.

In certain embodiments, where the cationic functional group of the (e.g., dually) derivatized-chitosan polyplex is an amino moiety, the compositions comprising chitosan polyplex complexed with the anionic portion-containing polymer can be characterized by the ratio of amino groups of the (e.g., dually) derivatized-chitosan polyplex (N) to anion (A) moieties of the polymer, referred to as the “N:A molar ratio.” This N:A molar ratio can vary from greater than about 1:100 to less than about 10:1.

In certain embodiments, the N:A molar ratio can be from greater than about 1:75 to less than about 8:1. In some cases, the N:A molar ratio can be from greater than 1:10 to less than 10:1. In some cases, the N:A molar ratio can be from, or from about, 1:10 to, or to about, 10:1. In some cases, the N:A molar ratio can be from, or from about, 1:8 to, or to about, 8:1. In certain embodiments, the N:A molar ratio can be from greater than 1:50 to less than about 10:1. In some cases, the N:A molar ratio can be from greater than 1:25 to less than about 10:1. In some cases, the N:A molar ratio can be from greater than 1:10 to less than about 7:1. In some cases, the N:A molar ratio can be from greater than 1:8 to less than about 7:1. In some cases, the N:A molar ratio can be from greater than 1:8 to less than about 6:1.

Additionally or alternatively, the compositions comprising chitosan polyplex complexed with the anionic portion-containing polymer can be characterized by a three-component ratio of cationic functional groups of the (e.g., dually) derivatized-chitosan polyplex (+) to phosphorus atoms of the nucleic acid (P) to anion moieties of the polymer (−), referred to as the “(+):P:(−) molar ratio.”

In certain embodiments, where (+):P is from at least 2:1 to no more than 20:1, the molar ratio of (+):(−) can vary from at least 1:40 to about 40:1. In certain embodiments, where (+):P is from at least 2:1 to no more than 20:1, the molar ratio of (+):(−) can vary from at least 1:40 to about 1:10. In some embodiments, where (+):P is from at least 2:1 to no more than 20:1, the molar ratio of (+):(−) can vary from at least 1:25 to about 25:1. In some embodiments, where (+):P is from at least 2:1 to no more than 20:1, the molar ratio of (+):(−) can vary from at least 1:25 to about 1:10. In some cases, where (+):P is from at least 2:1 to no more than 20:1, the molar ratio of (+):(−) can vary from at least 1:20 to about 20:1. In some cases, where (+):P is from at least 2:1 to no more than 20:1, the molar ratio of (+):(−) can vary from at least 1:20 to about 1:10. In some cases, where (+):P is from at least 2:1 to no more than 20:1, the molar ratio of (+):(−) can vary from at least 1:10 to about 10:1. In some cases, where (+):P is from at least 2:1 to no more than 20:1, the molar ratio of (+):(−) can vary from at least 1:25 to about 2:1. In some cases, where (+):P is from at least 2:1 to no more than 20:1, the molar ratio of (+):(−) can vary from at least 1:20 to about 1:1.

In certain preferred embodiments, (+):P:(−) is from 3:1:3.5 to 3:1:17.5. In certain preferred embodiments, (+):P:(−) is from 5:1:3.5 to 5:1:17.5. In certain preferred embodiments, (+):P:(−) is from 7:1:3.5 to 7:1:17.5. In certain preferred embodiments, (+):P:(−) is about 3:1:3.5, 3:1:7, 3:1:10, 3:1:15, 3:1:17.5, or 3:1:20. In certain preferred embodiments, (+):P:(−) is about 5:1:3.5, 5:1:7, 5:1:10, 5:1:15, 5:1:17.5, or 5:1:20. In certain preferred embodiments, (+):P:(−) is about 7:1:3.5, 7:1:7, 7:1:10, 7:1:15, 7:1:17.5, or 7:1:20. In certain preferred embodiments, (+):P:(−) is about 10:1:10, 10:1:15, 10:1:20, 10:1:25, 10:1:30, or 10:1:40.

One of skill in the art will appreciate that amino-functionalized chitosan polyplex particles in complex with the anionic portion-containing polymer can be characterized by a three-component ratio of amino functional groups of the (e.g., dually) derivatized-chitosan polyplex (N) to phosphorus atoms of the nucleic acid (P) to anion moieties of the polymer (A), referred to as the “N:P:A molar ratio.” In certain embodiments, where N:P is from at least 2:1 to no more than 20:1, the molar ratio of P:A can vary from at least 1:40 to about 40:1.

In certain embodiments, where N:P is from at least 2:1 to no more than 20:1, the molar ratio of P:A can vary from at least 1:40 to about 1:10. In certain embodiments, where N:P is from at least 2:1 to no more than 20:1, the molar ratio of P:A can vary from at least 1:25 to about 25:1. In certain embodiments, where N:P is from at least 2:1 to no more than 20:1, the molar ratio of P:A can vary from at least 1:25 to about 1:10. In some cases, where N:P is from at least 2:1 to no more than 20:1, the molar ratio of P:A can vary from at least 1:20 to about 20:1. In some cases, where N:P is from at least 2:1 to no more than 20:1, the molar ratio of P:A can vary from at least 1:20 to about 1:10. In some cases, where N:P is from at least 2:1 to no more than 20:1, the molar ratio of P:A can vary from at least 1:10 to about 10:1. In some cases, where N:P is from at least 2:1 to no more than 20:1, the molar ratio of P:A can vary from at least 1:25 to about 2:1. In some cases, where N:P is from at least 2:1 to no more than 20:1, the molar ratio of P:A can vary from at least 1:20 to about 1:1.

In certain preferred embodiments, N:P:A is from 3:1:3.5 to 3:1:17.5. In certain preferred embodiments, N:P:A is from 5:1:3.5 to 5:1:17.5. In certain preferred embodiments, N:P:A is from 7:1:3.5 to 7:1:17.5. In certain preferred embodiments, N:P:A is from 10:1:10 to 10:1:40. In certain preferred embodiments, N:P:A is about 3:1:3.5, 3:1:7, 3:1:10, 3:1:15, 3:1:17.5, or 3:1:20. In certain preferred embodiments, N:P:A is about 5:1:3.5, 5:1:7, 5:1:10, 5:1:15, 5:1:17.5, or 5:1:20. In certain preferred embodiments, N:P:A is about 7:1:3.5, 7:1:7, 7:1:10, 7:1:15, 7:1:17.5, or 7:1:20. In certain embodiment, N:P:A is about 10:1:10, 10:1:15, 10:1:20, 10:1:25, 10:1:30 or 10:1:40.

### C.1 Hydrophilic Non-charged Portion

The hydrophilic non-charged portion of the polymer can be, or comprise, a polyalkylene polyol or a polyalkyleneoxy polyol portion, or combinations thereof. The hydrophilic non-charged portion of the polymer can be, or comprise, a polyalkylene glycol or polyalkyleneoxy glycol portion. In certain embodiments, the polyalkylene glycol portion is or comprises a polyethylene glycol portion and/or a monomethoxy polyethylene glycol portion. In certain preferred embodiments, the non-charged portion of the polymer is, or comprises polyethylene glycol. The hydrophilic non-charged portion of the polymer can be, or comprise, other biologically compatible polymer(s) such as polylactic acid.

In addition to PEG, several hydrophilic non-charged entities are known in the art. For example, see: Lowe et.al., Antibiofouling polymer interfaces: poly(ethyleneglycol) and other promising candidates, Polym. Chem., 6, 198-212, 2015, and Knop et.al., Poly(ethylene glycol) in Drug Delivery: Pros and Cons as Well as Potential Alternatives. Angewandte Chemie International Edition, 49(36), 6288-6308, 2010. Examples of hydrophilic non-charged portion of the polymer are but not limited to: poly(glycerol), poly(2-methacryloyloxyethyl phosphorylcholine), poly(sulfobetaine methacrylate), and poly(carboxybetaine methacrylate), poly(2-methyl-2-oxazoline), poly(2-ethyl-2-oxazoline), and poly(vinylpyrrolidone).

The hydrophilic portion can have a weight average molecular weight of from about 500 Da to about 50,000 Da. In some embodiments, the hydrophilic portion has a weight average molecular weight of from about 1,000 Da to about 10,000 Da. In certain embodiments, the hydrophilic portion has a weight average molecular weight of from about 1,500 Da to about 7,500 Da. In certain embodiments, the hydrophilic portion has a weight average molecular weight of from about 3,000 Da to about 5,000 Da. In some cases, the hydrophilic portion has a weight average molecular weight of, or of about, 5,000 Da.

### C. 2 Anionic Polymer Portion

The anionic polymer portion of the polymer can comprise a plurality of functional groups that are negatively charged at physiological pH. A wide variety of anionic polymers are suitable for use in the methods and compositions described herein, provided that such anionic polymers can be provided as a component of a polymer having a hydrophilic non-charged polymer portion and are capable of forming a (e.g., reversible) charge:charge complex with the positively charged (e.g., dually) derivatized-chitosan-nucleic acid nanoparticles.

Exemplary anionic polymers include, but are not limited to, polypeptides having a net negative charge at physiological pH. In some cases, the polypeptides, or a portion thereof, consist of amino acids having a negatively charged side-chain at physiological pH. For example, the anionic polymer portion of the polymer can be a polyglutamate polypeptide, a polyaspartate polypeptide, or a mixture thereof. Additional amino acids, or mimetics thereof, can be incorporated into the polyanionic polypeptide. For example, glycine and/or serine amino acids can be incorporated to increase flexibility or reduce secondary structure.

In some cases, the anionic polymers can be or comprise an anionic carbohydrate polymer. Exemplary anionic carbohydrate polymers include, but are not limited to, glycosaminoglycans that are negatively charged at physiological pH. Exemplary anionic glycosaminoglycans include, but are not limited to, chondroitin sulfate, dermatan sulfate, keratin sulfate, heparin, heparin sulfate, hyaluronic acid, or a combination thereof. In certain embodiments, the anionic polymer portion of the polymer is or comprises hyaluronic acid.

Additional or alternative anionic carbohydrate polymers can include polymers comprising dextran sulfate.

In some cases, the polyanion portion is, or comprises, a polyanion selected from the group consisting of polymethacrylic acid and its salts, polyacrylic acid and its salts, copolymers of methacrylic acids and its salts, and copolymers of acrylic acid and/or methacrylic acid and its salts, such as a polyalkylene oxide, polyacrylic acid copolymer.

In some cases, the polyanion portion is, or comprises, a polyanion is selected from the group consisting of alginate, carrageenan, furcellaran, pectin, xanthan, hyaluronic acid, heparin, heparan sulfate, chondroitin sulfate, cellulose, oxidized cellulose, carboxymethyl cellulose, croscarmellose, synthetic polymers and copolymers containing pendant carboxyl groups, phosphate groups or sulphate groups, polyaminoacids of predominantly negative charge, and biocompatible polyphenolic materials.

The anionic portion of the polymers can have a weight average molecular weight of from about 500 Da to about 5,000 Da. In some embodiments, the anionic portion has a weight average molecular weight of from about 500 Da to about 3,000 Da. In certain embodiments, the anionic portion has a weight average molecular weight of from about 500 Da to about 2,500 Da. In certain embodiments, the anionic portion has a weight average molecular weight of from about 500 Da to about 2,000 Da. In certain embodiments, the anionic portion has a weight average molecular weight of from about 500 Da to about 1,500 Da. In some embodiments, the anionic portion has a weight average molecular weight of from about 1,000 Da to about 5,000 Da. In some embodiments, the anionic portion has a weight average molecular weight of from about 1,000 Da to about 3,000 Da. In certain embodiments, the anionic portion has a weight average molecular weight of from about 1,000 Da to about 2,500 Da. In certain embodiments, the anionic portion has a weight average molecular weight of from about 1,000 Da to about 2,000 Da. In some cases, the anionic portion has a weight average molecular weight of, or of about, 1,500 Da.

As used herein, “block copolymer,” “block co-polymer,” and the like refers to a copolymer containing distinct homopolymer regions. A diblock copolymer contains two distinct homopolymer regions. A triblock copolymer contains three distinct homopolymer regions. The three distinct regions can each be different (e.g., AAAA-BBBB-CCCC), or two regions can be the same (e.g., AAAA-BBBB-AAAA) similar (e.g., AAAA-BBBB-AAA), wherein “A,” “B,” and “C” represent different monomer subunits that form copolymer is comprised. For example, “A” can represent an ethylene glycol monomer subunit of a polyethylene glycol homopolymer and B can represent a glutamic acid subunit of a polyglutamic acid homopolymer. The block copolymer can be a linear (e.g., di- or tri-) block copolymer. Exemplary embodiments of linear diblock and triblock copolymers for use in the subject disclosure include those listed in the following non-exhaustive list:

PEG-Polyglutamic acid
methoxy-poly(ethylene glycol)-block-poly(L-glutamic acid)
mPEG*K-b-PLE##
mPEG1K-b-PLE10
mPEG1K-b-PLE50
mPEG1K-b-PLE100
mPEG1K-b-PLE200
mPEG5K-b-PLE10
mPEG5K-b-PLE50
mPEG5K-b-PLE100
mPEG5K-b-PLE200
mPEG10K-b-PLE10
mPEG10K-b-PLE50
mPEG10K-b-PLE100
mPEG10K-b-PLE200
mPEG20K-b-PLE10
mPEG20K-b-PLE50
mPEG20K-b-PLE100
mPEG20K-b-PLE200
PEG-Polyaspartic acid
methoxy-poly(ethylene glycol)-block-poly(L-aspartic acid)
mPEG*K-b-PLD##
mPEG1K-b-PLD10
mPEG1K-b-PLD50
mPEG1K-b-PLD100
mPEG1K-b-PLD200
mPEG5K-b-PLD10
mPEG5K-b-PLD50
mPEG5K-b-PLD100
mPEG5K-b-PLD200
mPEG20K-b-PLD10
mPEG20K-b-PLD50
mPEG20K-b-PLD100
mPEG20K-b-PLD200
PGA-PEG-PGA
poly(L-glutamic acid)-block-poly(ethylene glycol)-block-poly(L-glutamic acid)
PLE##-b-PEG*K-b-PLE##
PLE10-b-PEG1K-b-PLE10
PLE50-b-PEG1K-b-PLE50
PLE100-b-PEG1K-b-PLE100
PLE10-b-PEG5K-b-PLE10
PLE50-b-PEG5K-b-PLE50
PLE100-b-PEG5K-b-PLE100
Polyaspartic-PEG-polyaspartic
poly(L-aspartic acid)-block-poly(ethylene glycol)-block-poly(L-aspartic acid)
PLD##-b-PEG*K-b-PLD##
PLD10-b-PEG1K-b-PLD10
PLD50-b-PEG1K-b-PLD50
PLD100-b-PEG1K-b-PLD100
PLD10-b-PEG5K-b-PLD10
PLD50-b-PEG5K-b-PLD50
PLD100-b-PEG5K-b-PLD100
PEG- poly glutamic acid -PEG
Methoxy-poly(ethylene glycol)-block-poly(L- glutamic acid)-block-poly(ethylene glycol)
PEG*K-b-PGA##-b-PEG*K
PEG1K-b-PGA10-b-PEG1K
PEG1K-b-PGA50-b-PEG1K
PEG1K-b-PGA100-b-PEG1K
PEG5K-b-PGA10-b-PEG5K
PEG5K-b-PGA50-b-PEG5K
PEG5K-b-PGA100-b-PEG5K
PEG- polyaspartic-PEG
Methoxy-poly(ethylene glycol)-block-poly(L-aspartic acid)-block-poly(ethylene glycol)
PEG*K-b-PLD##-b-PEG*K
PEG1K-b-PLD10-b-PEG1K
PEG1K-b-PLD50-b-PEG1K
PEG1K-b-PLD100-b-PEG1K
PEG5K-b-PLD10-b-PEG5K
PEG5K-b-PLD50-b-PEG5K
PEG5K-b-PLD100-b-PEG5K
*K: molecular weight of PEG in kDa
##number of subunits

In one embodiment, the block copolymer is or comprises a PEG-polyglutamic acid polymer having the following structure:

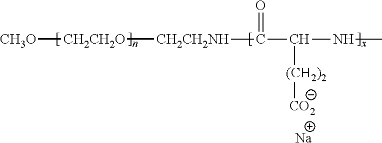

In one embodiment, the block copolymer is or comprises a PEG-polyaspartic acid polymer having the following structure:

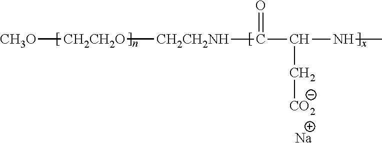

In one embodiment, the block copolymer is or comprises a PEG-hyaluronic acid polymer having the following structure:

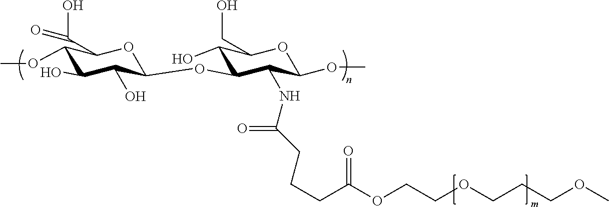

### D. Alternative Cationic Polymers and Lipids

The nucleic acid polyplexes of the subject disclosure function to condense and protect the nucleotides from enzymatic degradation. In addition to chitosan and derivatives thereof, alternative materials that can also be advantageously used for this purpose include other positively-charged (i.e. cationic) polymers and/or lipids.

Examples of cationic polymers that can be used to form polyplexes with the therapeutic nucleic acid constructs of the current disclosure include polyamines; polyorganic amines (e.g., polyethyleneimine (PEI), polyethyleneimine celluloses, and derivatives thereof); poly(amidoamines) (PAMAM and derivatives thereof); polyamino acids (e.g., polylysine (PLL), polyarginine, and derivatives thereof); polysaccharides (e.g., cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins. See, e.g., Samal et al., Cationic polymers and their therapeutic potential, Chem Soc Rev. 41:7147-94 (2012)

Examples of positively-charged lipids include esters of phosphatidic acid with an aminoalcohol, such as an ester of dipalmitoyl phosphatidic acid or distearoyl phosphatidic acid with hydroxyethylenediamine. More particular examples of positively charged lipids include 3β-[N--(N′,N′-dimethylaminoethyl)carbamoyl) cholesterol (DC-chol); N,N′-dimethyl-N,N′-dioctacyl ammonium bromide (DDAB); N,N′-dimethyl-N,N′-dioctacyl ammonium chloride (DDAC); 1,2-dioleoyloxypropyl-3-dimethyl-hydroxyethyl ammonium chloride (DORI); 1,2-dioleoyloxy-3-[trimethylammonio]-propane (DOTAP); N-(1-(2,3-dioleyloxy)propyI)-N,N,N-trimethylammonium chloride (DOTMA); dipalmitoylphosphatidylcholine (DPPC); 1,2-dioctadecyloxy-3-[trimethylammonio]-propane (DSTAP); and the cationic lipids described in e.g. Martin et al., Current Pharmaceutical Design 2005, 11, 375-394.

Blends of lipids and polymers in any concentration and in any ratio can also be used. Blending different polymer types in different ratios using various grades can result in characteristics that borrow from each of the contributing polymers. Various terminal group chemistries can also be adopted.

### III. Methods of Making

As described above, one of skill in the art will appreciate that polyplex:polymer particles of the disclosure may be produced by a variety of methods. For example, polyplex particles can be generated and then contacted with polymer. In an exemplary non-limiting embodiment, polyplex particles are prepared by providing and combining functionalized chitosan and nucleotide feedstock. Feedstock concentrations may be adjusted to accommodate various amino-to-phosphate ratios (N/P), mixing ratios and target nucleotide concentrations. In some embodiments, particularly small batches, e.g., batches under 2 mL, the functionalized chitosan and nucleotide feedstocks may be mixed by slowly dripping the nucleotide feedstock into the functionalized chitosan feedstock while vortexing the container. In other embodiments, the functionalized chitosan and nucleotide feedstocks may be mixed by in-line mixing the two fluid streams. In other embodiments, the resulting polyplex dispersion may be concentrated by means known in the art such as ultrafiltration (e.g., tangential flow filtration (TFF)), or solvent evaporation (e.g., lyophilization or spray drying). A preferred method for polyplex formation is disclosed in WO 2009/039657, which is expressly incorporated herein in its entirety by reference.

Similarly, polyplex particle feedstock (e.g., an aqueous solution comprising the polyplex compositions) can be provided (e.g., isolated from the reaction mixtures described above) and combined with polymer feedstock (e.g., an aqueous solution comprising the polymer). Feedstock concentrations may be adjusted to accommodate various amino-to-anion ratios (N/A), amino-to-phosphorous (N:P) ratios, N:P:A ratios, mixing ratios and target nucleotide concentrations. In some embodiments, particularly small batches, e.g., batches under 2 mL, the feedstocks may be mixed by slowly dripping a first feedstock (e.g., polyplex) into a second feedstock (e.g., polymer) while vortexing the container. In other embodiments, the feedstocks may be mixed by in-line mixing the two fluid streams. In other embodiments, the resulting polyplex:polymer complex dispersion may be concentrated by means known in the art such as ultrafiltration (e.g., tangential flow filtration (TFF)), or solvent evaporation (e.g., lyophilization or spray drying).

### 1. Powdered Formulations

The polyplex:polymer compositions of the disclosure include powders. In a preferred embodiment, the disclosure provides a dry powder polyplex:polymer composition. In a preferred embodiment, the dry powder polyplex:polymer composition is produced through the dehydration (e.g., spray drying or lyophilization) of a chitosan-nucleic acid polyplex dispersion of the disclosure.

### 2. Pharmaceutical Formulations

The present disclosure also provides “pharmaceutically acceptable” or “physiologically acceptable” formulations comprising polyplex:polymer compositions of the disclosure. Such formulations can be administered in vivo to a subject in order to practice the disclosed treatment methods.

As used herein, the terms “pharmaceutically acceptable” and “physiologically acceptable” refer to carriers, diluents, excipients and the like that can be administered to a subject, preferably without producing excessive adverse side-effects (e.g., nausea, abdominal pain, headaches, etc.). Such preparations for administration include sterile aqueous or non-aqueous solutions, suspensions, and emulsions. Liquid formulations include suspensions, solutions, syrups and elixirs. Liquid formulations may be prepared by the reconstitution of a solid.

Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like.

Excipients can include a salt, an isotonic agent, a serum protein, a buffer or other pH-controlling agent, an anti-oxidant, a thickener, an uncharged polymer, a preservative or a cryoprotectant. Excipients used in compositions of the disclosure may further include an isotonic agent and a buffer or other pH-controlling agent. These excipients may be added for the attainment of preferred ranges of pH (about 6.0-8.0) and osmolarity (about 50-400 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. Preferably, the isotonic agent is glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the disclosure may further contain a preservative. Examples preservatives are polyhexamethylene-biguanidine, benzalkonium chloride, stabilized oxychloro complexes (such as those known as Purite®), phenylmercuric acetate, chlorobutanol, sorbic acid, chlorhexidine, benzyl alcohol, parabens, and thimerosal. Typically, such preservatives are present at concentrations from about 0.001 to 1.0%. Furthermore, the compositions of the disclosure may also contain a cryopreservative agent. Preferred cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 10%.

A pharmaceutical formulation can be formulated to be compatible with its intended route of administration. For example, for oral administration, a composition can be incorporated with excipients and used in the form of tablets, troches, capsules, e.g., gelatin capsules, or coatings, e.g., enteric coatings (Eudragit® or Sureteric®). Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or other stearates; a glidant such as colloidal silicon dioxide; a sweetening agent such as sucrose or saccharin; or a flavoring agent such as peppermint, methyl salicylate, or flavoring.

Formulations can also include carriers to protect the composition against rapid degradation or elimination from the body, such as a controlled release formulation, including implants and microencapsulated delivery systems. For example, a time delay material such as glyceryl monostearate or glyceryl stearate alone, or in combination with a wax, may be employed.

Suppositories and other rectally administrable formulations (e.g., those administrable by enema) are also contemplated. Further regarding rectal delivery, see, for example, Song et al., Mucosal drug delivery: membranes, methodologies, and applications, Crit. Rev. Ther. Drug. Carrier Syst., 21:195-256, 2004; Wearley, Recent progress in protein and peptide delivery by noninvasive routes, Crit. Rev. Ther. Drug. Carrier Syst., 8:331-394, 1991.

Additional pharmaceutical formulations appropriate for administration are known in the art and are applicable in the methods and compositions of the disclosure (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

### IV. Administration

In one embodiment, the use of polyplexes:polymer compositions provides for prolonged stability of polyplexes at physiological pH. This provides for effective administration.

Any of a number of administration routes to contact cells, or tissue are possible and the choice of a particular route will in part depend on the target cell or tissue. Syringes, endoscopes, cannulas, intubation tubes, catheters, nebulizers, inhalers and other articles may be used for administration.

In some embodiments, the cancer is bladder cancer. Intravesical administration of chemotherapeutic agents is standard care for some bladder cancers. Briefly, intravesical therapy involves instillation of a therapeutic agent directly into the bladder via insertion of a urethral catheter. In some embodiments, the subject compositions provide for enhanced stability in urine, thereby improving localized expression.

The doses or “effective amount” for treating a subject are preferably sufficient to ameliorate one, several or all of the symptoms of the condition, to a measurable or detectable extent, although preventing or inhibiting a progression or worsening of the disorder or condition, or a symptom, is a satisfactory outcome. Thus, in the case of a condition or disorder treatable by expressing a therapeutic nucleic acid in target tissue, the amount of therapeutic RNA or therapeutic protein produced to ameliorate a condition treatable by a method of the disclosure will depend on the condition and the desired outcome and can be readily ascertained by the skilled artisan. Appropriate amounts will depend upon the condition treated, the therapeutic effect desired, as well as the individual subject (e.g., the bioavailability within the subject, gender, age, etc.). The effective amount can be ascertained by measuring relevant physiological effects.

Veterinary applications are also contemplated by the present disclosure. Accordingly, in one embodiment, the disclosure provides methods of treating non-human mammals, which involve administering a polyplex:polymer composition of the disclosure to a non-human mammal in need of treatment. The compositions of the disclosure may also be administered to the mucosa. For example, the compositions can be administered to mucosal cells or tissue of the gastrointestinal tract, including but not limited to mucosal cells or tissues of the small intestine and/or large intestine. Other target mucosal cells or tissues include, but are not limited to ocular, airway epithelial, lung, vaginal, and bladder cells or tissues. Other target cells or tissues include, but are not limited to cells of the breast, colon, prostate, pancreas, skin, lung, ovaries, kidney, brain, bladder, vagina, cervix, stomach, gastrointestinal tract, kidney, liver, thyroid, esophagous, nasal cancer, larynx, oral cancer, pharyngeal cancer, retinoblastoma, endometrium, and testicals, etc.

Typical formulations for this purpose include liquids, gels, hydrogels, solutions, creams, foams, films, implants, sponges, fibers, powders, and microemulsions.

The compounds of the disclosure can be administered to the mucosa intranasally or by inhalation, typically in the form of a dry powder (either alone, as a mixture, for example, in a dry blend with lactose, or as a mixed component particle) from a dry powder inhaler or as an aerosol spray from a pressurized container, pump, spray, atomizer, or nebulizer, with or without the use of a suitable propellant.

Capsules, blisters and cartridges for use in an inhaler or insufflator may be formulated to contain a powder mix of the compound of the disclosure, a suitable powder base such as lactose or starch and a performance modifier such as I-leucine, mannitol, or magnesium stearate.

Formulations for inhaled/intranasal administration may be formulated to be immediate and/or modified release. Modified release formulations include delayed-, sustained-, pulsed-, controlled-, targeted and programmed release.

The compounds of the disclosure may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the disclosure may also be administered directly to the eye or ear, typically in the form of drops. Other formulations suitable for ocular and aural administration include ointments, biodegradable (e.g. absorbable gel sponges, collagen) and non-biodegradable (e.g. silicone) implants, wafers, lenses and particulate systems. Formulations may also be delivered by iontophoresis.

Formulations for ocular/aural administration may be formulated to be immediate and/or modified release. Modified release formulations include delayed, sustained, pulsed, controlled, targeted, or programmed release.

### 1. Mucosal Administration

In some embodiments, the compositions of the disclosure are administered to the mucosa. For example, the compositions can be administered to mucosal cells or tissue of the bladder and gastrointestinal tract, including but not limited to mucosal cells or tissues of the small intestine and/or large intestine and/or colon. Other target mucosal cells or tissues include, but are not limited to ocular, airway epithelial, lung, vaginal, and bladder cells or tissues.

Typical formulations for this purpose include liquids, gels, hydrogels, solutions, creams, foams, films, implants, sponges, fibres, powders, and microemulsions.

In an exemplary embodiment for the bladder mucosa, the compounds described herein can be administered using intravesical therapy. Intravesical therapy involves instillation of a therapeutic agent directly into the bladder via insertion of a urethral catheter. The agent is allowed to sit in the bladder for a period of time, between 0.5 and 6 hours. It is a standard route of administration for bladder cancer chemotherapies. It utilizes the outside anatomical access available for drug delivery directly to the disease site in bladder and thereby avoids unwanted exposure of the instilled drug to healthy tissues elsewhere in the body.

Formulations for bladder administration may be formulated to be immediate and/or modified release. Modified release formulations include delayed-, sustained-, pulsed-, controlled-, targeted, or programmed release

The compounds of the disclosure can also be administered to the mucosa intranasally or by inhalation, typically in the form of a dry powder (either alone, as a mixture, for example, in a dry blend with lactose, or as a mixed component particle) from a dry powder inhaler or as an aerosol spray from a pressurized container, pump, spray, atomiser, or nebuliser, with or without the use of a suitable propellant.

Capsules, blisters and cartridges for use in an inhaler or insufflator may be formulated to contain a powder mix of the compound of the disclosure, a suitable powder base such as lactose or starch and a performance modifier such as I-leucine, mannitol, or magnesium stearate.

Formulations for inhaled/intranasal administration may be formulated to be immediate and/or modified release. Modified release formulations include delayed-, sustained-, pulsed-, controlled-, targeted and programmed release.

The compounds of the disclosure may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the disclosure may also be administered directly to the eye or ear, typically in the form of drops. Other formulations suitable for ocular and aural administration include ointments, biodegradable (e.g. absorbable gel sponges, collagen) and non-biodegradable (e.g. silicone) implants, wafers, lenses and particulate systems. Formulations may also be delivered by iontophoresis.

Formulations for ocular/aural administration may be formulated to be immediate and/or modified release. Modified release formulations include delayed-, sustained-, pulsed-, controlled-, targeted, or programmed release.

### 2. Intratumoral Administration

In some embodiments, the compositions of the disclosure are administered directly to a tumor (a cancer). For example, the compositions can be administered to a cancer in a tissue such as the breast, prostate, skin, lung, brain, bladder, stomach, kidney, etc., by directly contacting and locally administering a composition comprising a nucleic acid polyplex comprising a cationic polymer and/or lipid, a therapeutic nucleic acid construct encoding interleukin-12 (IL-12), and a therapeutic nucleic acid construct comprising a nucleic acid encoding at least one RIG-I agonist, wherein the therapeutic nucleic acid constructs encoding IL-12 and RIG-I are the same or different nucleic acid constructs, to the cancer.

Intratumoral injection is known in the art (see e.g., Melero et al., (2021) Nature Reviews Clinical Oncology 18: 558-576).

### V. Therapeutic Applications

The methods disclosed herein activate a strong memory T cell response to a cancer antigen. Accordingly, therapeutic proteins contemplated for use in the disclosure have a wide variety of activities and find use in the treatment of a wide variety of disorders. Thus, the following description of therapeutic protein activities, and indications treatable with therapeutic nucleic acids and proteins of the disclosure, is exemplary and not intended to be exhaustive. The term “subject” refers to an animal, with mammals being preferred, and humans being especially preferred. Specific non-limiting examples of therapeutic embodiments are described below.

In some therapeutic embodiments, the therapeutic polyplex:polymer composition is applied directly to a tumor e.g., by intratumoral injection. Where the therapeutic effect applies to metastatic disease, the cells or tissues contacted by the polyplex:polymer compositions described herein are tumoral, but the therapeutic effect is distal to the primary tumor or primary target tissue.

In some cases, the therapeutic embodiments are applied to mucosal tissue, but are intended to act on non-mucosal target tissues, cells, or organs. In such embodiments, where the therapeutic effect is non-mucosal, it is understood that the cells or tissues contacted by the polyplex:polymer compositions described herein are mucosal. In some embodiments the therapeutic action is proximal to the mucosal target. For example, mucosal cells can be transfected to produce and secrete IL-12 and/or another immunostimulatory molecule. However, in other embodiments where the therapeutic effect is non-mucosal, as in metastatic disease, the cells or tissues contacted by the polyplex:polymer compositions described herein are mucosal, but the therapeutic effect is distal to the primary tumor or primary mucosal target tissue.

In embodiments, polyplex:polymer compositions of the disclosure may be used for therapeutic treatment or prophylactic treatment. Such compositions are sometimes referred to herein as therapeutic compositions. As noted above, the subject compositions and methods primarily employ therapeutic nucleic acids encoding IL-12, either alone or in conjunction with additional innate and/or adaptive immunostimulatory molecules such as e.g., a RIG-I agonist. In some embodiments, the therapeutic nucleic acid further encodes an IFN-1 activator/inducer such as, e.g., a RIG-I agonist, a STING agonist, a TLR 7/9 agonist, and/or other Pattern Recognition Receptor agonists. See, e.g. Vasou et al., Viruses 9:186 (2017). In some embodiments, the therapeutic nucleic acid further encodes a modulator of an immune checkpoint molecule selected from the group consisting of CTLA-4, PD-1, PD-L1, PD-L2, TIM3, B7-H3, B7-H4, LAG-3, KIR, and ligands thereof.

Suitable IFN-1 activator/inducers include RIG-I agonists (such as eRNA11a, adenovirus VA RNA1, eRNA41H, MK4621 (Merck), SLR10, SLR14, and SLR20), STING (i.e., stimulator of interferon genes) agonists (such as CDN, i.e., cyclic dinucleotides), PRRago (such as CpG, Imiquimod, or Poly I:C), and TLR agonists (such as CPG-1826, GS-9620, AED-1419, CYT-003-QbG10, AVE-0675, or PF-7909) including TRL7 and TLR9, and RLR stimulators (such as RIG-I, Mda5, or LGP2 stimulators). In some embodiments, the IFN-1 activator/inducer induces dendritic cells, T cells, B cells, and/or T follicular helper cells.

In preferred embodiments, the IFN-1 activator/inducer is a RIG-I agonist. RIG-I (retinoic acid inducible gene I, encoded by Ddx58) is a cytosolic antiviral helicase that acts as an RNA sensor, detecting and being activated upon recognition of viral RNAs in the cytoplasm. A pattern recognition receptor, RIG-I contains an RNA helicase domain and two N-terminal caspase recruitment domains (CARDs), which relay a signal to the downstream signaling adaptor MAVS (mitochondrial antiviral-signaling protein). RIG-1 signaling via MAVS leads to a variety of responses including induction of type I IFN responses, including IFNα and IFNβ, via TBK1 and IRF7/8, and activation of caspase-8-dependent apoptosis. They are found in most tissues, including cancer cells (Kato et al., Immunol. Rev.243(1):91-98 (2011)).

RIG-I induced responses differs between cells. While normal healthy cells such as melanocytes and fibroblasts are quite resistant to RIG-I-induced apoptosis, tumor cells are highly susceptible to RIG-I-induced cell death (Besch et al., 2009; Kubler et al., 2010). RIG-I's natural ligands are viral short blunt ends of duplex RNA containing 5′tri or diphosphate (5′ppp or 5′pp). RIG-I-specific ligands are currently being developed for immunotherapy of cancer (Duewell et al., 2014, 2015; Ellermeier et al., 2013; Schnurr & Duewell, Oncoimmunology, 2(5):e24170 (2013) and 2014). Part of the potent antitumor activity of RIG-I ligands is the downstream ability to promote cross-presentation of antigens to CD8⁺ cells and to induce cytotoxic activity (Hochheiser et al., 2016). RIG-I ligands also show strong therapeutic activity in viral infection models such as influenza (Weber-Gerlach & Weber, 2016).

Plasmid vector backbones expressing RIG-I ligands from RNA polymerase III promoters have been used to identify potent synthetic RIG-I ligands (Luke et al., J. Virol. 85(3):1370-1383). Stem-loop RNA modified with tri-phosphate are of particular use as agonists in the instant disclosure. These include, but are not limited to, eRNA41H, which combines (i) eRNA11a, an immunostimulatory dsRNA expressed by convergent transcription, with (ii) adenovirus VA RNAI, SLR20, a double-stranded, triphosphorylated 20-base pair stem-loop RNA, modified with a 5′ triphosphate sequence (Elion et al., Cancer Res. 78(21):6183-6195 (2018)), and SLR10 and SLR14, which are alternative polyphosphorylated RNAs with a stable tetraloop at one end (Jiang et al., J. Exp. Med. 216:2854-68 (2019)).

Additional RIG-I agonists finding advantageous use in the compositions and methods described herein include SB-9200, a broad-spectrum antiviral innate sensor agonist that acts via the activation of the RIG-I and nucleotide-binding oligomerization domain 2 pathway (Jones et al. J. Med. Virol. 89:1620-1628 (2017), MK 4621 (RGT100, Merck), CBS-13-BPS, a synthetic RIG-I-specific agonist mimicking the structure of the influenza virus panhandle promoter (Lee et al. Nucleic Acids Res. 46:10553 (2018); IVT-B2 RNA (Lien et al. Molecular Therapy 24:135-45 (2016), SeV DVGs (Xu et al., mBio 65:e01265-15 (2015)), 5′ppp RNA with uridine-rich sequence with 99 nucleotides hairpin (M8) (Chiang et al. J. Virol. 89:8011-25 (2015), and 3pRNA.

In accordance with the foregoing embodiments, RIG-I agonists suitable for co-expression with IL-12 in the subject compositions and methods include, but are not limited to: RIG-I DNA vaccines, plasmid encoded RNA polymerase III expressed RNA-based RIG-I agonists such as, e.g., eRNA11a, adenovirus VA RNA1, eRNA41H (Nature Technology Corp), GFP2, Lamin A/C and Lamin VSV, tri-GFPs, SAD ΔPLp, Tri-G-AC-U, Flu vRNA, RNaseL fragments, pppRVL, pppVSVL, ppp-shRNA-luc3VA1, 5′ppp-dsRNA, 3p-hpRNA, MK4621 (Merck), SLR10, SLR14, SLR20, CBS-13-BPS, IVT-B2 RNA, SeV CVG, SB-9200, and siRNAs as disclosed in Ellermeier et al., Cancer Research (2013) 73(6). Similarly, STING agonists suitable for coexpression with IL-12 include, but are not limited to: DExD/H helicases including DDX41, and TLR agonists include, but are limited to CpG dinucleotides such as, e.g., CpG-1826 (ODN1826, Invivogen).

In accordance with the foregoing embodiments, modulators of immune checkpoint molecules suitable for coexpression with IL-12 in the subject compositions and methods include, e.g., single domain antibodies (sdAb) directed to one or more of CTLA-4, PD-1, PD-L1, PD-L2, TIM3, B7-H3, B7-H4, LAG-3, and KIR (such as, e.g., KN035 (Ablynx/Sanofi); Inhibrix 105), (see also Wan et al., Oncol. Rep. (2018); Hosseinzadeh et al., Rep. Biochem & Mol. Bio., (2017); Dougan et al., Can. Imm. Res. (2016); Ingram et al., PNAS (2018), and WO2017198212); dominant negative PD-1 molecules (e.g., Atara Therapeutics), PD-1 variants having high affinity for PD-L1 (e.g. competitive antagonists) (Maute, PNAS (2015)); and CD80 variant(s) with increased binding to CD28 (e.g. WO2017/181152).

In some cases, said IFN-1 agonist and/or said immune checkpoint inhibitor is encoded by:
said therapeutic nucleic acid construct in said derivatized chitosan nucleic acid polyplex;
a different therapeutic nucleic acid construct in said derivatized chitosan nucleic acid polyplex;
a therapeutic nucleic acid construct in a different derivatized chitosan nucleic acid polyplex (e.g., that does not comprise a construct encoding IL-12);
a therapeutic nucleic acid construct (e.g., formulated in an alternate nucleic acid delivery formulation, such as a PEI or cationic lipid formulation).

The therapeutic nucleic acid construct encoding IL-12 and the therapeutic nucleic acid construct encoding said IFN-1 agonist and/or said immune checkpoint inhibitor can be simultaneously or sequentially administered. In some cases, the therapeutic nucleic acid construct encoding said IFN-1 agonist and/or said immune checkpoint inhibitor are co-administered in a single formulation or in single, e.g., admixed, combination of two different formulations. In some cases, the therapeutic nucleic acid construct encoding IL-12 and the therapeutic nucleic acid construct encoding said IFN-1 agonist and/or said immune checkpoint inhibitor are administered sequentially.

The immunostimulatory molecule of the disclosure may also encode an shRNA (short hairpin RNA) molecule designed to inhibit protein(s) involved in the growth or maintenance of tumor cells or other hyperproliferative cells. A plasmid DNA may simultaneously encode for a therapeutic protein and one or more shRNA. Furthermore, the nucleic acid of the said composition may also be a mixture of plasmid DNA and synthetic RNA including sense RNA, antisense RNA or ribozymes.

### VI. Methods of Treatment

### Hyperproliferative Disorders

The subject compositions and methods find advantageous use in the treatment of hyperproliferative disorders. Exemplary hyperproliferative disorders include hyperproliferative disorders of the breast, colon, prostate, pancreas, skin, lung, ovary, kidney, brain, bladder, vagina, cervix, stomach, gastrointestinal tract, kidney, liver, thyroid, esophagous, nasal, laryx, oral, pharyx, retina, endometrium, testes, etc. Of particular interest are compositions and methods for the treatment of hyperproliferative disorders that have metastasized from a primary cancer/tumor to a site distinct from the primary cancer.

In some embodiments, the hyperproliferative disorder is a hyperproliferative disorder mucosal tissues or in tissues proximal to mucosal tissue. Methods and compositions of the disclosure may be used in the treatment of gastrointestinal cancers including, but not limited to oral cancers, esophageal cancers, stomach cancers, pancreatic cancers, liver cancers, colorectal cancers, and rectal cancers. Nasal and pulmonary cancers which may be treated by the methods and compositions of the disclosure include, but are not limited to, paranasal sinus cancer, oropharyngeal cancer, tracheal cancer, and lung cancers. Genitourinary cancers which may be treated by the methods and compositions of the disclosure include, but are not limited to bladder cancers, urothelial cancers, urethral cancers, testicular cancers, kidney cancers, prostate cancers, penile cancers, adrenal cancers, uterine cancers, cervical cancers and ovarian cancers.

In some embodiments according to any one of the methods provided above, the method further comprises administering (such as systemically or locally to the site of the tumor) a non-nucleic acid-based immunostimulatory molecule.

In some embodiments, the immunostimulatory molecule is a modulator of an immune checkpoint molecule selected from the group consisting of CTLA-4, PD-1, PD-L1, PD-L2. TIM3, B7-H3, B7-H4, LAG-3, KIR, and ligands thereof. In some embodiments, the immunomodulator is an inhibitor of PD-L1 or PD-L1. In some embodiments, the inhibitor of PD-1 is an anti-PD-1 antibody, such as pembrolizumab or nivolumab. In some embodiments, the immunomodulator is an inhibitor of CTLA-4. In some embodiments, the inhibitor of CTLA-4 is an anti-CTLA-4 antibody, such as ipilimumab or tremelimumab. In some embodiments, the inhibitor of PD-L1 is an anti-PD-L1 antibody, such as atezolizumab.

In some embodiments, the immunomodulator is an IFN-1 agonist, e.g. a RIG-I agonist, a STING agonist, or a TLR 7/9 agonist. RIG-I agonists suitable for co-administration include, but are not limited to short poly I:C and polyAU compositions (e.g. Poly(I:C)/Lyo Vec complexes (Invivogen)); RGT100 (MK4621, Merck) SLR20 (Elion et al.; SLR10 & SLR14 (Jiang et al.);; and agonists as disclosed in U.S. Pat. Nos. 8,871,799, 8,895,608, 8,927,561, 9,073,946, 9,458,492, 9,555,106, 9,884,876, 9,956,285, 9,775,894, 9,861,574, 9,937,247, 10,167,476, 10,350,158, 10,434,064, 10,273,484, 9,381,208B2, 9,738,680B2, 9,790,509, 10,059,943, 9,109,012B2, 9,937,247B2, 9,816,091B2, 9,133,456B2, 9,409,941B2, 9,340,789B2, 9,040,234B2, US 20200071316, US20200063141A1, US20200061097A1, US20200055871A1, US20200016253A1, US20190076463A1, US20180195063A1, US20160287623A1.

STING agonists suitable for co-administration in conjunction with IL-12 include, but are not limited to c-Di-AMP sodium salt, c-Di-GMP sodium salt, 2′,3′-cGAMP sodium salt, 3′,3′-cGAMP sodium salt, 10-carboxymethyl-9-acridanone (CMA), DMXAA (Tocris Bioscience, InvivoGen, Nimbus Therapeutics), G10, α-Mangostin, CRD100 (Curadev), cAIMP, 2′2′-c-GAMP, 2′3′-cGAM(PS)2(Rp/Sp), 2′3′-c-di-AMP, c-di-IMP, c-di-UMP, 5,6-dimethylxanthenone-4-acetic acid (DMXAA), MK-1454 (Merck) ML RR-S2 CDG, ML RR-S2 CDA (ADU-S100), SB11285 (Springbank Pharmaceuticals), MAVU (AbbVie), DiABZI, disodium dithio-(Rp1Rp)-[cyclic[A(2′5′)pA(3′5′)p]][Rp,Rp]-cyclic9adenosine-(2′5′)-monophosphorothioate-adenosine-(3′5′)-monophosphorothioate), disodium (RR-S2 CDA, ADU-S100, MIW815) (Corrales et al.,2016) and the compositions disclosed in U.S. Pat. Nos. 10,176,292, 9,724,408, 10,011,630, 10,435,469, 10,414,747, 10,413,612, 10,131,686, 10,106,574, 10,047,115, 10,045,961, 10,011,630, 9,994,607, 9,937,247, 9,840,533, 9,770,467, 9,724,408, 9,718,848, and 9,642,830.

TLR7 and TLR9 agonists suitable for co-administration with IL-12 include, but are not limited to: imidazoquinolines and their analogs, including Resiquimod and Imiquimod (Aldara), hydroxycholoroquine, chloroquire, bropirimine, Loxoribine, Isatoribine, CpG oligonucleotides, stabilized immune modulatory RNA (SIMRA) AST-008 (Exicure), MEDI9197 and the compositions disclosed in U.S. Pat. Nos. 434,064, 10,413,612, 10,407,431, 10,370,342, 10,364,266, 10, 208,037, 10,202,386, 9,944,649, 9,902,730, 9,868,955, 9,359,360, 9,295,732, 9,243,050, 9,228,184, 9,216,192, 9,2206,430, 8,735,421, 8,728,486, 8,399,423 and 8,242,106.

In some embodiments, the non-nucleic acid-based immunomodulator and the subject compositions are administered simultaneously, such as in the same composition. In some embodiments, the non-nucleic acid-based immunomodulator and the subject compositions are administered sequentially.

In some embodiments, the methods for treating cancer provided herein further comprise administering to the subject at least one additional therapeutic agent. In further embodiments, the additional therapeutic agent is a chemotherapeutic drug or a radiotherapeutic drug. In some embodiments, the chemotherapeutic drugs include, but are not limited to, cisplatin, carboplatin, paclitaxel, docetaxel, 5-fluorouraci 1, bleomycin, methotrexate, ifosamide, oxaliplatin, cyclophosphamide, dacarbazine, temozolomide, gemcitabine, capecitabine, cladribine, clofarabine, cytarabine, floxuridine, fludarabine, hydroxyurea, pemetrexed, pentostatin, thioguanadine, daunorubicin, doxurubicin, epirubicin, idarubicin, topotecan, irinotecan, etoposide, eniposide, colchicine, vincristine, vinblastine, and vinorelbine. Exemplary cancer specific agents and antibodies include, but are not limited to, Afatinib, Aldesleukin, Alemtuzumab, Axitinib, Belimumab, Bevacizumab, Bortezomib, Bosutinib, Brentuximab vedotin, Cabozantinib, Canakinumab, Carfilzomib, Cetuximab, Crizotinib, Dabrafenib, Dasatinib, Denosumab, Erlotinib, Everolimus, Gefitinib, lbritumomab tiuxetan, lbrutinib, Imatinib, Ipilimumab, Lapatinib, Nilotinib, Obinutuzumab, Ofatumumab, Panitumumab, Pazopanib, Pertuzumab, Ponatinib, Regorafenib, Rituximab, Romidepsin, Ruxolitinib, Sipuleucel-T, Sorafenib, Temsirolimus, Tocilizumab, Tofacitinib, Tositumomab, Trametinib, Trastuzumab, Vandetanib, Vemurafenib, Vismodegib, Vorinostat, Ziv-aflibercept, and any combination thereof. In some embodiments, the additional therapeutic agent is administered to the subject prior to, concurrently with, or subsequent to administration of the immunoconjugate. In some embodiments, the additional therapeutic agent is administered systemically. For example, in some embodiments, the additional therapeutic agent is administered by intravenous injection.

In some embodiments, the cancer treated using the methods disclosed herein is bladder cancer. The conventional bladder cancer treatment currently approved in the U.S. is intra-urethral Bacillus Calmette-Guerin vaccine. This antigenic vaccine is thought to stimulate bladder cells to express interferon, which in turn recruits the patient's innate immune system to better recognize cancer cell surface antigens and attack cancer cells. In over a third of cases, however, the vaccine is ineffective. Similarly, intravesical instillation of exogenously manufactured interferon polypeptide has also been tested, but has not been effective. The subject compositions and methods can also be advantageously employed in conjunction with these more conventional approaches to augment and improve the immune response.

The examples set out herein illustrate several embodiments of the present disclosure but should not be construed as limiting the scope of the present disclosure in any manner.

### EXAMPLES

### Example 1: Assessment of Durable Anti-Tumor Immunity in an Orthotopic Model of Bladder Cancer

To evaluate the durable anti-tumor immunity of mEG-70 nanoparticles, an orthotopic model of murine bladder cancer was used. Briefly, the codon optimized murine IL-12 (opt-mouse IL-12p40p35) gene encodes the two sub-units (p40 and p35) of the cytokine protein, IL-12. To ensure 1:1 stoichiometry of the subunits, the mEG-70 plasmid was designed to contain a single open reading frame (ORF) to monomerize p40 to p35 by the addition of a short repeating elastin linker sequence. The codon-optimized sequence was cloned into the NTC₉₃₈₅R or NTC₉₃₈₅R-eRNA41H vector backbones and expression was confirmed as disclosed in as disclosed in WO2020/183239, which is incorporated by reference herein in its entirety. The plasmids comprise genes for eRNA11a (an immunostimulatory double-stranded ribonucleic acid [dsRNA]) and adenovirus VA RNA1. The two RNA products of these genes stimulate the RIG-I pathway, which recruits more immune cells to the local tissue.

This therapeutic nucleic acid is packaged in a dually-derivatized chitosan polymer functionalized with arginine and glucose and coated with a detachable PEG-b-PLE excipients, to form the pharmaceutical composition mEG-70 as disclosed in WO2020/183239.

Disease was established by pretreating murine bladders with poly-L-lysine to promote desquamation of the superficial urothelial layer and facilitate cancer cell implantation. Urothelial carcinoma cells that stably overexpress the luciferase gene (MB49-Luc) were subsequently instilled into murine bladders (100,000 cells per mouse) and luciferase expression was confirmed at Day 9 post-instillation using an In Vivo Imaging System (IVIS). The intensity of the bioluminescent signal was used to randomize animals into treatment groups (n=22) based on the level of bioluminescence. Animals without a positive bioluminescent signal were excluded from the study.

Mice received an intravesical instillation (IVI) of mEG-70 (1 mg DNA/mL; equivalent to 80 μg DNA) under anesthesia with isoflurane, on Day 10 (Tx1) and Day 17 (Tx2), with control animals receiving an instillation of 1% mannitol (sham). This dosing regimen was selected based on the assessment of protein expression kinetics determined as disclosed in WO 2020/183239. A cohort of tumor-bearing animals was untreated.

Survival was monitored for 85 days following instillation of MB49-Luc cells. Statistical significance was analysed by log-rank (Mantel-Cox) test (*p<0.05 and **p<0.01 for mEG-70 relative to sham and untreated, respectively). (C) Surviving tumor-free mEG-70-treated mice (negative bioluminescence signal, no clinical signs), and age-matched controls, were rechallenged by IVI of MB49-Luc cells (1×10⁵ cells) on Day 85. Tumor implantation was monitored by in vivo imaging of bioluminescence at 7, 14 and 21 days following the rechallenge (Study Days 92, 99, and 106, respectively).

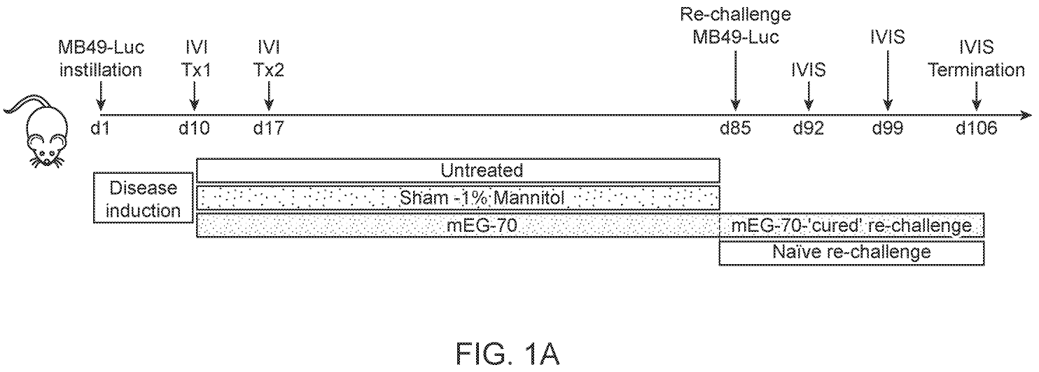

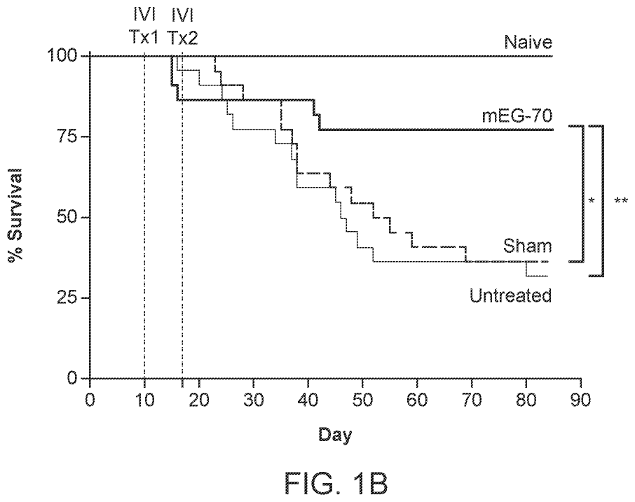

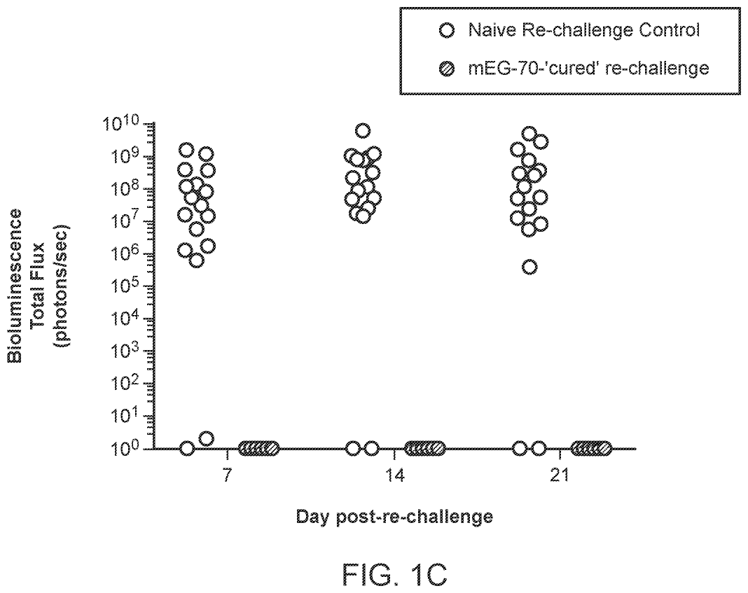

As shown in FIG. 1B, mEG-70-treated animals exhibited long-term survival compared to control mice, of which approximately 70% succumbed to disease. The survival curve for mEG70 is significantly different from the survival of sham-treated (1% mannitol) or untreated mice (*p<0.05 and **p<0.01, respectively).

Treated mice that demonstrated complete disease regression and did not relapse during the 76-day observation period (referred to as ‘mEG-70 cured’), were re-challenged with MB49-Luc cells to assess protection from recurring disease. In contrast to age-matched naïve controls, which showed robust tumor implantation in 15 out of 17 mice, all mEG-70-cured mice were resistant to tumor recurrence up to 3 weeks after re-challenge (n=17). See FIG. 1C.

Together these data suggest that durable, systemic anti-tumor immunity has been established in response to mEG-70 treatment.

### Example 2: Distant Tumor Re-Challenge

MB49-Luciferase cells (MB49-Luc; 1×10⁵ cells) were instilled into female C₅₇BL/6J bladders (12-16 weeks) as described in Example 1, and implantation was confirmed by in vivo imaging of luciferase signal at Day 9 post instillation (using the Lumina LT IVIS imaging system). Mice were distributed equally to treatment groups (n=22) based on the level of bioluminescence (luciferase negative mice were excluded from the study) and received an intravesical instillation (IVI) of mEG-70 nanoparticles (1 mg DNA/mL; equivalent to 80 μg DNA, see Example 1) on Day 10 (Tx1) and Day 17 (Tx2), with control animals receiving an instillation of 1% mannitol (sham). A cohort of tumor-bearing animals was untreated.

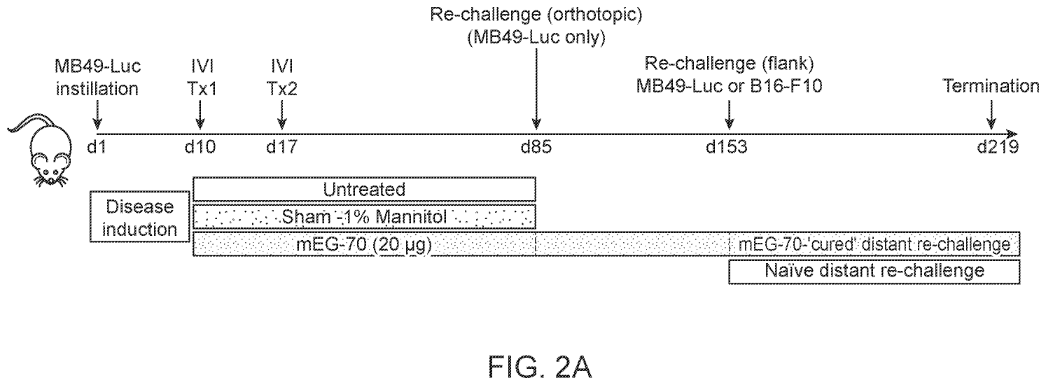

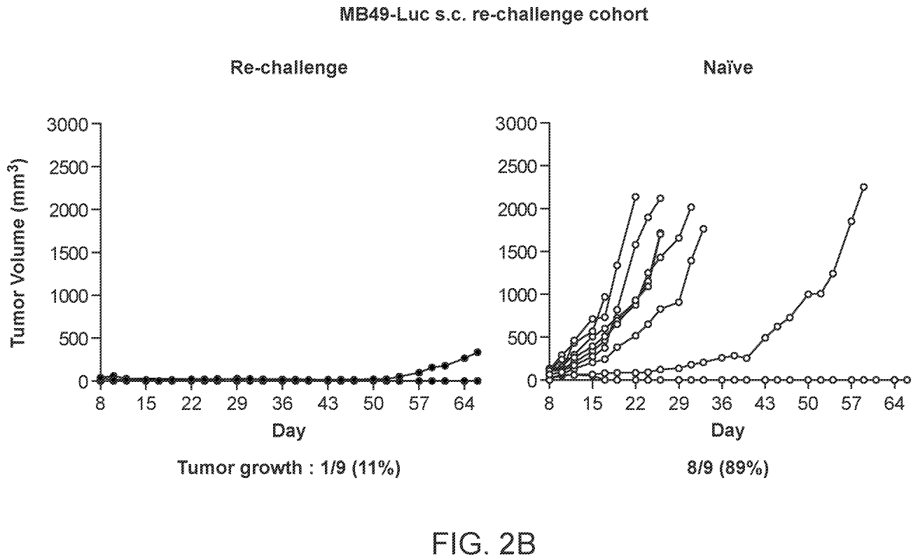

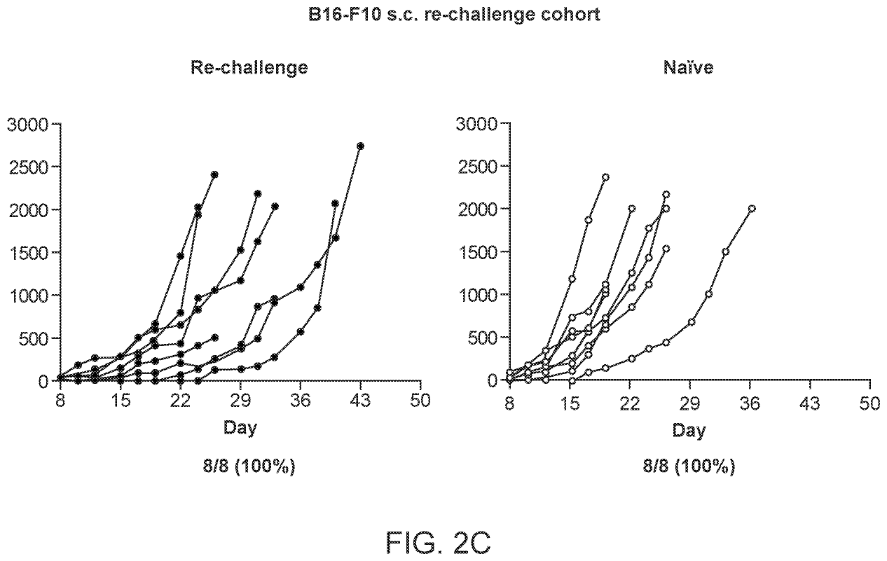

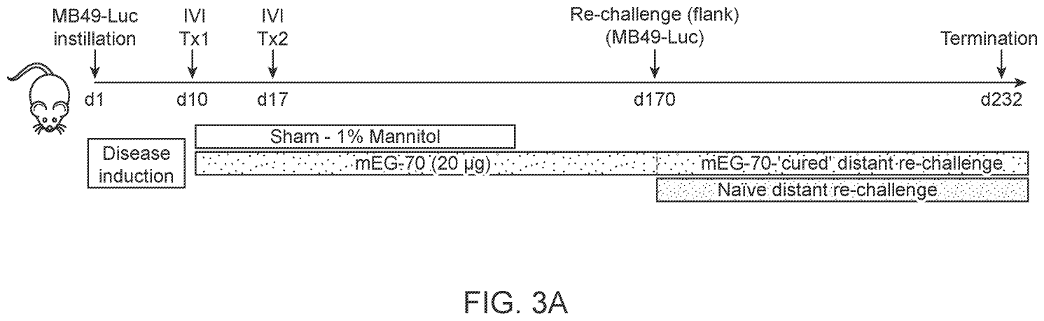

Survival was monitored until all mice succumbed to bladder cancer or were considered tumor-free (negative bioluminescence signal, no clinical signs). On Day 85, surviving tumor-free mEG-70-treated mice and age-matched controls, were re-challenged by IVI of MB49-Luc cells (1×10⁵ cells). All mEG-70-treated mice remained tumor-free and, on Day 153, were re-challenged subcutaneously on the flank with either MB49-Luc (1×10⁵ cells; FIG. 2B) or B16-F10 cells (1×10⁵ cells; FIG. 2C). A cohort of age-matched animals was also included to control for subcutaneous cell implantation. Tumors were monitored by measuring with a caliper; tumor volume was calculated using the formula (length×width²/2).

As shown in FIG. 2B, mEG-70-treated animals were protected from distant tumor re-challenge with MB49-Luc cells. Only 1 out of 9 animals showed tumor growth, which was markedly delayed. In contrast, the naïve control cohort had 8/9 mice with tumor growth.

Mice were re-challenged with B16-F10 cells to assess the specificity of the response. All mice from the re-challenged and naïve control group showed robust B16-F10 tumor implantation (n=8/group). See FIG. 2C.

Together these data suggest that durable, systemic and specific anti-tumor immunity has been established in response to mEG-70 treatment.

### Example 3: T Cell Depletion During Distal Tumor Re-Challenge

The following Example illustrates that the durable, systemic, anti-tumor immunity achieved with the subject therapy is T-cell dependent.

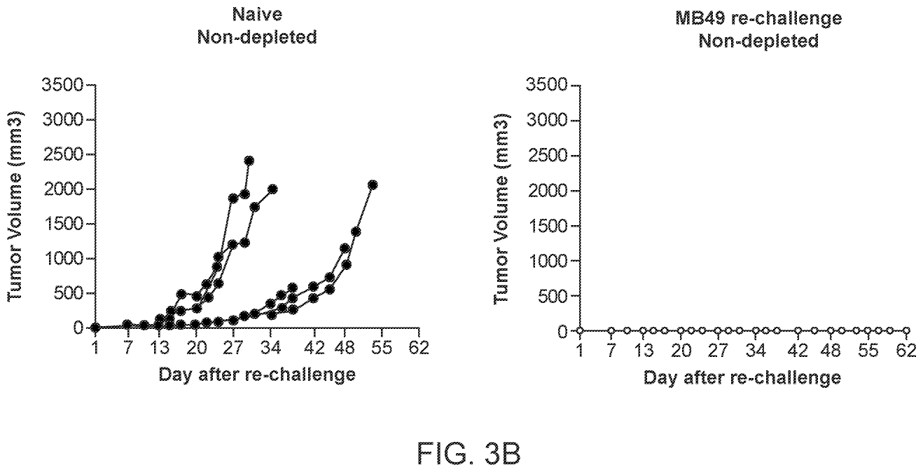

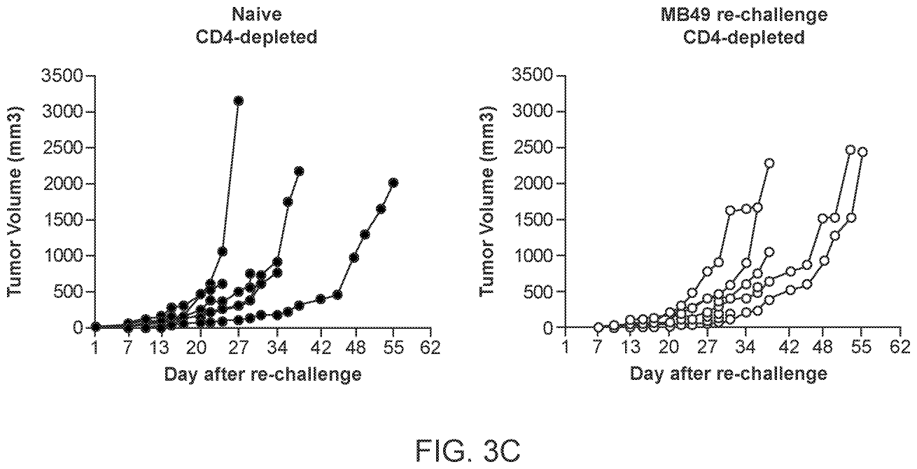

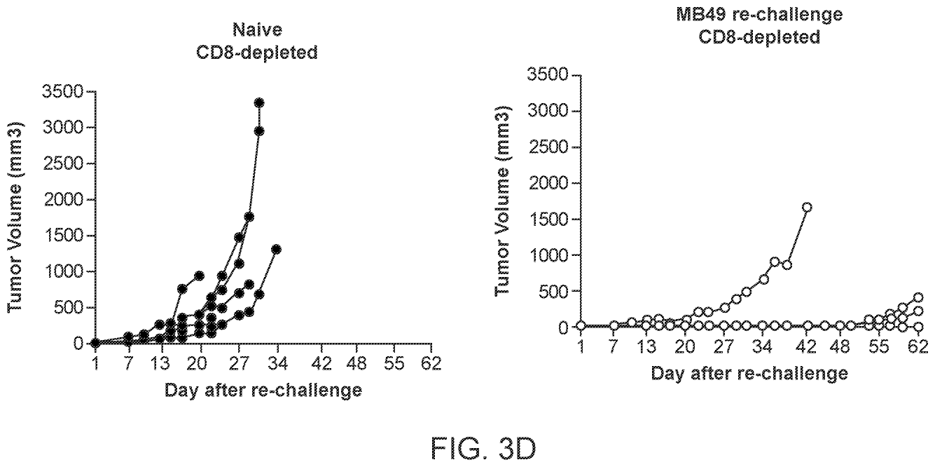

MB49-Luciferase cells (MB49-Luc; 1×10⁵ cells) were instilled into female C₅₇BL/6J bladders (12-16 weeks) as described in Example 1, and implantation was confirmed by in vivo imaging of luciferase signal at Day 9 post instillation (using the Lumina LT IVIS imaging system). Mice were distributed equally to treatment groups (n=20) based on the level of bioluminescence (luciferase negative mice were excluded from the study). FIG. 3A provides a schematic representation of the experimental timeline.

Mice received an intravesical instillation (IVI) of mEG-70 nanoparticles (1 mg DNA/mL; equivalent to 80 μg DNA, see Example 1) on Day 10 (Tx1) and Day 17 (Tx2), with control animals receiving an instillation of 1% mannitol (sham). Survival was monitored until all mice succumbed to bladder cancer or were considered tumor-free (negative bioluminescence signal, no clinical signs; data not shown).

On Day 167, surviving tumor-free mEG-70-treated mice, and age-matched naïve controls, were injected intraperitoneally with isotype control (non-depleted), anti-CD4 antibody, or anti-CD8 antibody for 4 consecutive days to establish depletion and then were injected twice a week to maintain. Mice were re-challenged subcutaneously on the flank with MB49-Luc cells (1×10⁵ cells) after the third depleting antibody injection (Day 170; n=6). Tumors were monitored by measuring with a caliper. Tumor volume was calculated using the formula [length×width²/2]. The results are summarized below and shown in FIG. 3 (B-D).

All naïve mice that received isotype control antibody (non-depleted) had a growing subcutaneous tumor. In contrast, mEG-70-treated mice were all protected from distant tumor re-challenge with the MB49-Luc cells (FIG. 3B).

All mice that received the anti-CD4 antibody (CD4+ T cell-depleted) whether naïve or previously cured by mEG-70 treatment, had a growing MB49-Luc subcutaneous tumor (FIG. 3C).

All naïve mice that received the anti-CD8 antibody (CD8+ T cell-depleted) had a growing subcutaneous tumor. In contrast, only 1 out of 6 mEG-70-treated animals had an actively growing tumor (FIG. 3D). Notably, 2 out of 6 CD8+ T cell-depleted, mEG-70-treated mice had a small tumor mass that was quiescent and had delayed growth.

Thus, protection from re-challenge in mEG-70-treated mice is impaired in the absence of CD4+ T cells. Accordingly, mEG-70 treatment provides a durable and systemic anti-tumor immunity that is largely mediated by CD4+ T cells.

### Example 4: Contralateral Flank Re-Challenge

The following Example illustrates that mEG-70, administered by direct intratumoral injection in subcutaneous tumors, results in anti-tumor response that is both durable and systemic.

MB49-Luciferase cells (MB49-Luc; 2.5×10⁵ cells in 100 μL) were implanted subcutaneously onto the right flank of C₅₇BL/6J mice (12-16 weeks) under anesthesia. When tumors reached ˜50-200 mm³, mice were randomized to treatment groups (n=10).

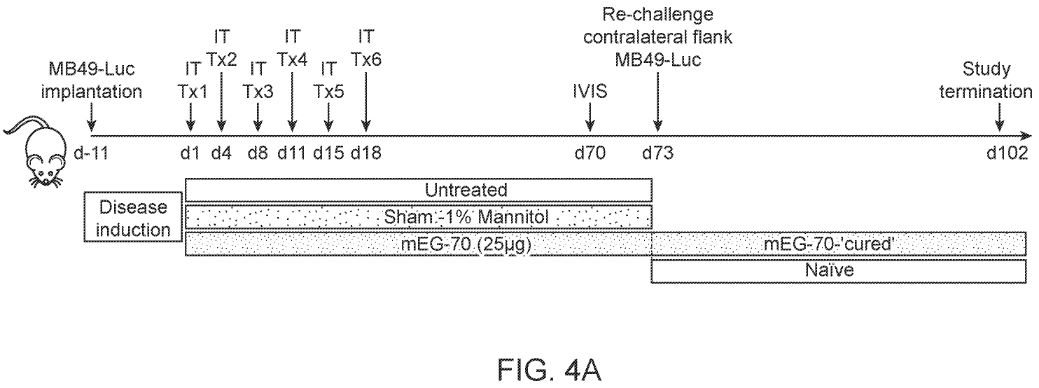

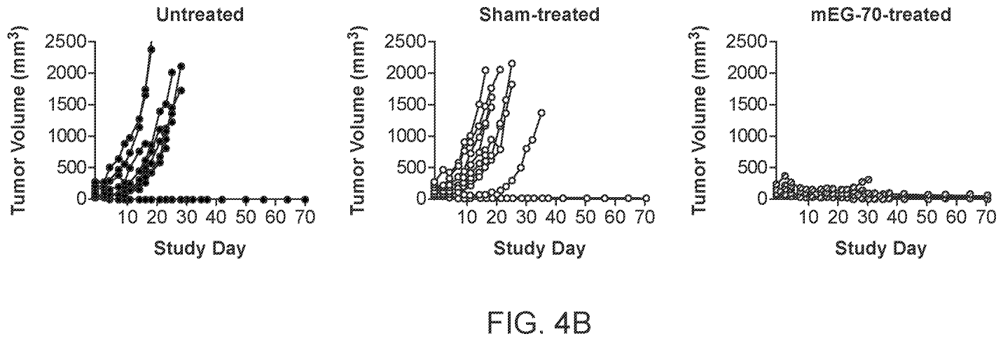

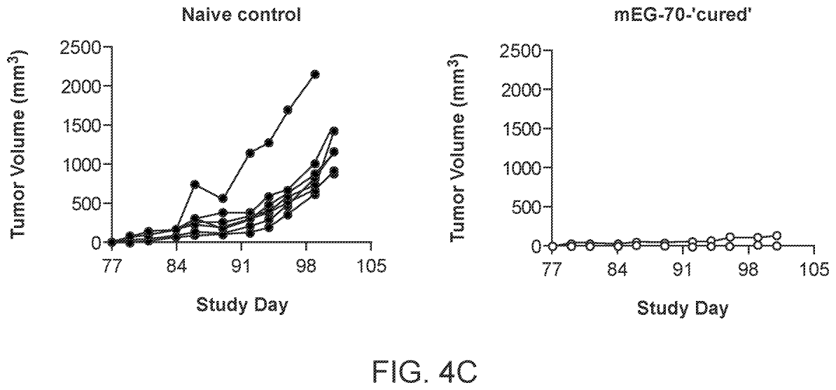

Mice received direct intratumoral (IT) administration of mEG-70 nanoparticles (0.5 mg DNA/mL in 50 μL; equivalent to 25 μg DNA) on Day 1, 4, 8, 11, 15 and 18 with control animals administered 1% mannitol (sham). A cohort of tumor-bearing animals was untreated. Tumor size was monitored by measuring with a caliper 3 times per week and tumor volume was calculated using the formula [length×width²/2] (FIG. 4A).

To confirm that tumors had not relapsed, bioluminescence imaging of luciferase signal was conducted on Day 70 in tumor-free mEG-70-treated mice (mEG-70-“cured”; n=9) using the Lumina LT IVIS imaging system. On Day 73, mEG-70-cured and age-matched controls, received subcutaneous implantation of MB49-Luc cells (2.5×10⁵ cells in 100 μL) on the left flank. Tumors were monitored three times per week by measuring with a caliper; tumor volume was calculated using the formula [length×width²/2].

As shown in FIG. 4B, intratumoral (IT) administration of mEG-70 inhibited tumor growth as compared to sham-treated mice. Furthermore, mEG-70-“cured” mice were protected from tumor cell re-challenge on the contralateral flank (FIG. 4C).

Accordingly, mEG-70 administered by direct intratumoral injection in subcutaneous tumors resulted in an anti-tumor response that is durable and systemic.

### Example 5: Human Clinical Study in Metastatic Bladder Cancer

Bladder cancer is the fourth and tenth most common malignancy among men and women in the United States (US), respectively (American Cancer Society 2019). Non-muscle invasive bladder cancer (NIMBC) is generally managed with surgical resection (TURBT) followed often by a single dose of intravesical chemotherapy within 24 hours (gemcitabine or mitomycin) to reduce the recurrence rate by 35% (Sylvester et al, 2016).

After confirmation of the presence of bladder cancer from pathology, physicians develop a continued treatment plan, frequently involving BCG therapy. Despite significant adverse effects, and a 30% to 40% failure rate, intravesical immunotherapy with BCG is the mainstay treatment used to prevent recurrence and/or progression in patients with high grade (Ta and above) NMIBC. BCG is often given in a second maintenance course to achieve a disease-free state, even though patients with BCG-unresponsive NMIBC are extremely unlikely to benefit from further therapy with BCG, and therefore represent a unique population for the study of new therapies (Jarow et al, 2015).

In the absence of pharmacologic intervention or cystectomy, BCG-unresponsive NMIBC, with or without resected disease, will persist and progress. To date, there are no effective therapies available for patients who have failed BCG, as gemcitabine and mitomycin often given post TURBT are not effective salvage agents. Therefore, the treatment for BGC-unresponsive disease (regardless if BCG refractory or relapsed) is radical cystectomy to surgically remove all tumor and ensure disease-free survival. The fact that there are few treatment options available for NMIBC, and patients continue to have radical organ removal for early stage disease describes a truly great unmet medical need. More effective treatments that are active in refractory patients are desperately needed in NMIBC.

In an exemplary embodiment, the therapeutic nucleic acid comprises a 4156 bp plasmid DNA (pDNA) comprised of a codon optimized human interleukin-12 gene termed opt-hIL-12 linked to a constitutively active cytomegalovirus (CMV) promoter on a NTC₉₃₈₅R backbone with an antibiotic-free selection marker based on sucrose (RNA-OUT), as set forth in SEQ ID NO: 8.

The R6K origin of replication restricts plasmid replication to a specific strain of Escherichia coli (E. coli). The opt-hIL12 gene encodes the two sub-units (p40 and p35) of the cytokine protein, IL-12. To ensure 1:1 stoichiometry of the subunits, the EG-70 plasmid was designed to contain a single open reading frame (ORF) to monomerize p40 to p35 by the addition of a short repeating elastin linker sequence. The plasmid is also comprised of genes for eRNA11a (an immunostimulatory double-stranded ribonucleic acid [dsRNA]) and adenovirus VA RNA1. The two RNA products of these genes stimulate the RIG-I pathway, which recruits more immune cells to the local tissue. In a further embodiment, this therapeutic nucleic acid is packaged in a dually-derivatized chitosan polymer functionalized with arginine and glucose and coated with a detachable PEG-b-PLE excipients, to form the pharmaceutical composition EG-70. The composition is formulated as an aqueous nanoparticle dispersion in 1% w/w mannitol solution, filter sterilized, lyophilized to a dry powder, and stored at 4° C. The average particle size of the nanoparticle dispersion is in the 75-175 nanometer range.

This study will evaluate the safety of intravesical administration of EG-70 and its effect on bladder tumors at distant sites in patients who have failed BCG therapy and are awaiting radical cystectomy. The study will be a classic dose escalation trial where 3 patients are treated in each cohort. The initial dose of EG-70 will be based on the nonclinical toxicology data as well as the nonclinical efficacy data, and will be at least ⅕ of the minimal toxic dose seen in the GLP-toxicology study. Projected Phase I dose escalations will be in up to ½-log increments for successive cohorts treated without dose-limiting toxicity (DLT).

Patients will be monitored to evaluate if delivery of nanoparticles to the bladder is sufficient to prime the immune system to prevent/eradicate growth of secondary tumors.

### EQUIVALENTS

All publications, patents, and patent applications mentioned in this specification are herein incorporated by reference in the entirety and for all purposes and to the same extent as if each individual publication, patent, or patent application was specifically and individually indicated to be incorporated by reference. The disclosure set forth above may encompass multiple distinct disclosures with independent utility. Although each of these disclosures has been disclosed in its preferred form(s), the specific embodiments thereof as disclosed and illustrated herein are not to be considered in a limiting sense, because numerous variations are possible. The subject matter of the disclosures includes all novel and nonobvious combinations and subcombinations of the various elements, features, functions, and/or properties disclosed herein. The following claims particularly point out certain combinations and subcombinations regarded as novel and nonobvious. Disclosures embodied in other combinations and subcombinations of features, functions, elements, and/or properties may be claimed in this application, in applications claiming priority from this application, or in related applications. Such claims, whether directed to a different disclosure or to the same disclosure, and whether broader, narrower, equal, or different in scope in comparison to the original claims, also are regarded as included within the subject matter of the disclosures of the present disclosure.

1. A method for activating a memory T cell response to a primary cancer in a patient in need thereof, the method comprising:
contacting a primary cancer in said patient with a therapeutically effective amount of a composition comprising a nucleic acid polyplex comprising a cationic polymer and/or lipid, a therapeutic nucleic acid construct encoding interleukin-12 (IL-12), and a therapeutic nucleic acid construct comprising a nucleic acid encoding at least one RIG-I agonist, wherein the therapeutic nucleic acid constructs encoding IL-12 and RIG-I are the same or different nucleic acid constructs.

2. The method of claim 1, wherein the method is effective for treating or suppressing a primary cancer in said patient other than a mucosal tumor.

3. The method of claim 1 or 2, wherein the method is effective for treating or suppressing metastatic disease in said patient at a site distinct from the primary cancer.

4. The method of any one of claims 1 to 3, wherein the primary cancer is selected from breast cancer, colon cancer, prostate cancer, pancreatic cancer, melanoma, lung cancer, ovarian cancer, kidney cancer, brain cancer, a sarcoma, bladder cancer, vaginal cancer, cervical cancer, stomach cancer, gastrointestinal cancer, kidney cancer, liver cancer, thyroid cancer, esophageal cancer, nasal cancer, laryngeal cancer, oral cancer, pharyngeal cancer, retinoblastoma, endometrial cancer, and testicular cancer.

5. The method of claim 3 or 4, wherein the site distinct from the primary cancer is one or more of: liver, lung, bone, brain, lymph node, peritoneum, skin, prostate, breast, colon, rectum, and cervix.

6. The method of any one of claims 3 to 5, wherein the metastatic disease is at two or more sites distinct from the primary cancer.

7. The method of any one of claims 1 to 6, wherein said RIG-I agonist is selected from the group consisting of eRNA11a, VA RNA1, eRNA41H, MK4621, SLR10, SLR14, and SLR20, and more preferably selected from the group consisting of eRNA41H, eRNA11a.

8. The method of any one of claims 1 to 7, wherein said cationic polymer is selected from the group consisting of polyethyleneimine (PEI), PAMAM, polylysine (PLL), polyarginine, chitosan, and derivatives thereof.

9. The method of claim 8, wherein the cationic polymer comprises a derivatized chitosan, preferably an amino-functionalized chitosan.

10. The method of claim 9, wherein said amino-functionalized chitosan comprises arginine and further comprises, or is functionalized with, a hydrophilic polyol.

11. The method of claim 10, wherein the hydrophilic polyol is selected from gluconic acid and glucose.

12. The method according to any one of the preceding claims, wherein the nucleic acid polyplex further comprises a reversible coating comprising one or more polyanion-containing block co-polymers having at least one polyanionic anchor region and at least one hydrophilic tail region, preferably wherein the polyanion-containing block co-polymer is a linear diblock and/or triblock co-polymer.

13. The method according to any one of the preceding claims, wherein said therapeutic nucleic acid construct encoding IL-12, comprises SEQ ID NO: 8.

14. A method for treating or suppressing tumor metastasis at a site distinct from the site of a primary cancer in a patient in need thereof, the method comprising:
contacting the primary cancer and/or the tumor metastases in said patient with a therapeutically effective amount of a composition comprising a nucleic acid polyplex comprising a cationic polymer and/or lipid, a therapeutic nucleic acid construct encoding interleukin-12 (IL-12), and a therapeutic nucleic acid construct comprising a nucleic acid encoding at least one RIG-I agonist, wherein the therapeutic nucleic acid constructs encoding IL-12 and RIG-I are the same or different nucleic acid constructs.

15. The method of claim 14, wherein the cancer is selected from a breast cancer, colon cancer, prostate cancer, pancreatic cancer, melanoma, lung cancer, pulmonary cancer, ovarian cancer, kidney cancer, brain cancer, a sarcoma, bladder cancer, vaginal cancer, cervical cancer, stomach cancer, gastrointestinal cancer, kidney cancer, thyroid cancer, esophageal cancer, laryngeal cancer, oral cancer, pharyngeal cancer, retinoblastoma, endometrial cancer, and testicular cancer.

16. The method of claim 15, wherein the primary cancer is selected from the group consisting of a gastrointestinal cancer, a nasal or pulmonary cancer, and a genitourinary cancer.

17. The method of claim 16, wherein the primary cancer is a gastrointestinal cancer, selected from the group consisting of an oral cancer, an esophageal cancer, a stomach cancer, a pancreatic cancer, a liver cancer, a colorectal cancer, and a rectal cancer.

18. The method of claim 16, wherein the primary cancer is a nasal or pulmonary cancer selected from the group consisting of a paranasal sinus cancer, an oropharyngeal cancer, a tracheal cancer, and a lung cancer.

19. The method of claim 16, wherein the primary cancer is a genitourinary cancer selected from the group consisting of a bladder cancer, a urothelial cancer, a urethral cancer, a testicular cancer, a kidney cancer, a prostate cancer, a penile cancer, an adrenal cancer, a uterine cancer, a cervical cancer, and an ovarian cancer.

20. The method of claim 19, wherein the genitourinary cancer is bladder cancer.

21. The method of any one of claims 14 to 20, wherein the tumor metastatic site is at one or more of: liver, lung, bone, brain, lymph node, peritoneum, skin, prostate, breast, colon, rectum, and cervix.

22. The method of any one of claims 14 to 21, wherein the tumor metastasis is at two or more different sites.

23. The method of any one of claims 14 to 22, wherein said RIG-I agonist is selected from the group consisting of eRNA11a, VA RNA1, eRNA41H, MK4621, SLR10, SLR14, and SLR20, and more preferably selected from the group consisting of eRNA41H, eRNA11a.

24. The method of any one of claims 14 to 23, wherein said cationic polymer is selected from the group consisting of polyethyleneimine (PEI), PAMAM, polylysine (PLL), polyarginine, chitosan, and derivatives thereof.

25. The method of claim 24, wherein the cationic polymer comprises a derivatized chitosan, preferably an amino-functionalized chitosan.

26. The method of claim 25, wherein said amino-functionalized chitosan comprises arginine and further comprises, or is functionalized with, a hydrophilic polyol.

27. The method of claim 26, wherein the hydrophilic polyol is selected from gluconic acid and glucose.

28. The method according to any one of claims 14-27, wherein the nucleic acid polyplex further comprises a reversible coating comprising one or more polyanion-containing block co-polymers having at least one polyanionic anchor region and at least one hydrophilic tail region, preferably wherein the polyanion-containing block co-polymer is a linear diblock and/or triblock co-polymer.

29. The method according to any one of claims 14 to 28, wherein said therapeutic nucleic acid construct encoding IL-12 comprises SEQ ID NO: 8.

30. The method of claim 14, wherein the contacting comprises intravesical instillation.

31. The method of claim 14, wherein the contacting is oral dosage or intrarectal/intracolonic administration to the gastrointestinal tract (GIT).

32. The method of claim 14, wherein the contacting is by intratumoral injection.

33. The method of claim 14, wherein the contacting is intranasal or intratracheal administration to the lungs.
